(19) United States
(12) Patent Application Publication
(10) Pub. No.: US 2022/0148685 A1
(43) Pub. Date: May 12, 2022

(54) FREE ENERGY LANDSCAPE MODELING WITH PARALLEL PATHS

(71) Applicant: X Development LLC, Mountain View, CA (US)

(72) Inventor: Frank Russo, Sunnyvale, CA (US)

(21) Appl. No.: 17/092,910

(22) Filed: Nov. 9, 2020

(51) Int. Cl. G16C 20/50; G16C 20/30; G16C 20/10; G16B 5/00; G16B 15/00

(52) U.S. Cl. CPC G16C 20/50; G16C 20/30; G16B 15/00; G16B 5/00; G16C 20/10

(57) ABSTRACT

Embodiments include simulating the behavior of enzymatic reactions involving multiple substrates binding with an enzyme. Some embodiments may allow for modeling the reactions without requiring known values for kinetic rate constants for microsteps of the reaction. Embodiments may include computer-implemented methods. Methods may include initializing an initial free energy profile for a reaction. The initial free energy profile may include a plurality of initial waypoints. The methods may include generating a plurality of rate equations, each rate equation representing a component step of the plurality of component steps. Methods may include simulating an in silico behavior of the reaction. Simulating may include generating concentrations of the substrates using the plurality of rate equations. Simulating may include updating the plurality of initial waypoints to reduce a difference between the in silico behavior of the reaction and an expected behavior of the reaction, forming a new free energy profile comprising a plurality of updated waypoints.

### FIELD

The present disclosure relates to kinematic modeling of biochemical pathways, and in particular to general and scalable techniques for modeling in silico the kinetics of systems of connected biochemical reactions.

### BACKGROUND

A biological system such as a living cell, or a population of living cells, can be modeled in silico such that the response of the living cell(s) to a variety of experiments can be performed quickly and cheaply in simulation. Simulated experiments may be performed to develop an understanding of the interrelationships of various environmental and other factors on the development of the biological system and/or on the biological system's effect on its environment. For example, simulated experiments may be performed to determine a set of environmental conditions that increases a growth rate of the biological system or that increases a rate of production of a substance of interest (e.g., an antibody, a hormone, a protein, an enzyme) by the biological system. Additionally or alternatively, the simulated experiments may be performed to develop an understanding of the effect of changes in the composition of the biological system (e.g., changes in the genetic or epigenetic makeup of the biological system) on the development or behavior of the biological system and/or to develop an understanding of the effect on changes in protein structure on the functionality of such proteins. For example, simulated experiments may be performed to determine a change in the genome of the biological system that increases a growth rate of the biological system and/or that increases a rate of production of a substance of interest by the biological system.

Biological systems and the biochemical processes thereof are typically modeled using various equations or functions and include many parameters, each corresponding to (e.g., ‘modeling’) an aspect of the structure and/or function of the biological system or biochemical process. Of the biochemical processes that need to be modelled to both infer molecular mechanisms and predict biological responses, many are enzyme reactions typically described using a system of differential equations. Modeling enzyme reactions and enzymatic cascade networks, however, often requires the simulation of multiple associated reactions. This inevitably increases the complexity of the system of differential equations, and exponentially increases the number of free kinetic parameters required for the modeling. The increase in free kinetic parameters can make it difficult to constrain all parameters simultaneously using a limited amount of available experimental data and can result in the derivation of biochemical process models with limited predictive power. There is a need for improving upon the modeling of biochemical processes with generalizable and scalable techniques to reduce parameter dimensionality without reducing the topological complexity required to capture key kinetic features in the biochemical processes.

One particular area of interest in modeling is enzymatic reactions with two or more substrates binding with a single enzyme. An enzymatic reaction with a single enzyme and a single substrate may include several microscopic steps (“microsteps”), for example, the enzyme binding to a substrate, the enzyme:substrate complex transitioning to an enzyme:product complex, and the enzyme:product complex dissociating to form an enzyme and a product. Reactions with two or more substrates may introduce more complexity and unknowns into a model. For example, multiple substrates binding with an enzyme may introduce additional microsteps to consider. An enzyme may bind with a first substrate, and the resulting complex may bind with a second substrate. Each of these binding microsteps involves a rate equation with a rate constant. The enzyme may also bind with a second substrate and the resulting complex may bind with a second substrate. These two binding microsteps result in another two rate equations with rate constants. As a result of the two substrates binding with an enzyme, four rate equations and four rate constants are added. As the reverse microsteps are also possible, binding two substrates with an enzyme may result in an additional eight rate equations and eight rate constants. Each rate constant may not be known and may be difficult to observe experimentally. A conventional approach to modeling that requires values for the rate constants may then involve laborious experiments before modeling the multi-substrate enzymatic reaction. Accordingly, there is a need for improving upon modeling of enzymatic reactions with two or more substrates.

### SUMMARY

Some embodiments of the present invention may allow for simulating the behavior of enzymatic reactions involving multiple substrates binding with an enzyme. Some embodiments may allow for modeling the reactions without requiring as many known values for kinetic rate constants for microsteps as with other modeling methods. Some embodiments may also result in more accurate simulations of enzymatic reactions, in part by avoiding simplifying assumptions that are not realistic.

Some embodiments include a computer-implemented method. The method may include initializing an initial free energy profile for a reaction. The reaction may be part of a pathway or process in a system to be modeled. The initial free energy profile may include a plurality of initial waypoints. Each initial waypoint may represent a free energy state. The reaction may include an enzyme, a first substrate, and a second substrate. The reaction may include a plurality of component steps (e.g., microsteps). Each end of a component step of the plurality of component steps may correspond to an initial waypoint of the plurality of initial waypoints. A first initial waypoint of the plurality of initial waypoints may represent a first free energy state of a multi-substrate complex of the enzyme bound to the first substrate and to the second substrate.

The method may also include generating a plurality of rate equations. Each rate equation may represent a component step of the plurality of component steps. Each rate equation may include a forward rate constant or a reverse rate constant and one or more concentrations of molecules, the molecules including the enzyme, the first substrate, and the second substrate. The method may further include simulating an in silico behavior of the reaction. Simulating the reaction may be by generating concentrations of the first substrate and the second substrate using the plurality of rate equations. Simulating the reaction may also be by updating the plurality of initial waypoints to reduce a difference between the in silico behavior of the reaction and an expected behavior of the reaction, forming a new free energy profile comprising a plurality of updated waypoints.

In some embodiments, the difference may be measured based on a comparison between a simulated catalytic rate constant k_{cat} and a simulated Michaelis constant Kₘ of the in silico behavior of the reaction to an expected catalytic rate constant k_{cat} and an expected Michaelis constant Kₘof the expected behavior of the reaction.

In some embodiments, a first difference in free energy from a beginning of the initial free energy profile to an end of the initial free energy profile may correspond to an expected change in free energy for the reaction. A second difference in free energy from a beginning of the new free energy profile to an end of the new free energy profile may equal the first difference.

In some embodiments, the plurality of component steps may include a first binding component step and a second binding component step. The first binding component step may include binding between the enzyme and the first substrate to form an enzyme:first substrate complex. The second binding component step may include binding between the enzyme and the second substrate to form an enzyme:second substrate complex.

In some embodiments, the plurality of component steps may include a third binding component step and a fourth binding component step. The third binding component step may include binding between the enzyme:first substrate complex and the second substrate to form the multi-substrate complex. The fourth binding component step may include binding between the enzyme:second substrate and the first substrate.

In some embodiments, the plurality of component steps may include a transition component step, a first dissociation component step, a second dissociation component step, a third dissociation step, and a fourth dissociation step. The transition component step may include a chemical conversion of the multi-substrate complex to a multi-product complex including the enzyme, a first product, and a second product. The first dissociation component step may include a dissociation of the multi-product complex into a first complex including the enzyme and the first product. The second dissociation component step may include a dissociation of the multi-product complex into a second complex including the enzyme and the second product. The third dissociation step may include a dissociation of the first complex into the enzyme and the first product. The fourth dissociation step may include a dissociation of the second complex into the enzyme and the second product.

Some embodiments include a method that further includes determining one of the forward rate constant or the reverse rate constant for a rate equation of the plurality of rate equations using the free energy state of an end of a component step and the other rate constant of the forward rate constant or the reverse rate constant.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

Some embodiments of the present invention will be better understood in view of the following non-limiting figures, in which:

FIG. 1 shows an interaction system for configuring and using a simulation to facilitate subsequent experiment configurations according to various embodiments;

FIG. 2 shows a representation of modules representing distinct biological functions according to various embodiments;

FIG. 3 shows a simulation controller that dynamically integrates results generated by different types of models to simulate higher-level states and reactions according to various embodiments;

FIG. 4 shows a process for dynamically synthesizing results generated by multiple simulators to simulate higher-level results according to various embodiments;

FIG. 5 shows a module-specific simulation controller to simulate states and reactions according to various embodiments;

FIG. 6 shows a process for using a simulator to generate metabolite time-course data according to various embodiments;

FIG. 7 shows a typical enzymatic reaction according to various embodiments;

FIG. 8 shows a binding-dissociation step of a reaction according to various embodiments;

FIG. 9 shows a transition step of a reaction according to various embodiments;

FIG. 10 shows a binding component and a transition component according to various embodiments;

FIG. 11 shows a first standardized mathematical construct that may be used to model a single binding component (binding or dissociation) and a second standardized mathematical construct that may be used to model a single transition component according to various embodiments;

FIGS. 12A-12D show a process flow for configuring components and sets of standardized mathematical constructs using an interaction system to simulate the overall behavior of a system according to various embodiments;

FIG. 13 shows the free energy states for a typical enzyme reaction according to various embodiments;

FIG. 14 shows the reaction rate V determined for each simulation being plotted as a function of substrate concentration [S] according to various embodiments;

FIG. 15 shows a best-fit line curve can be generated to connect the data points of each simulation and graph the reaction rate V as a function of substrate concentration [S] according to various embodiments;

FIG. 16 shows a process flow for configuring components and sets of standardized mathematical constructs using an interaction system to simulate the overall behavior of a system according to various embodiments;

FIG. 17 shows how waypoints within a free energy ΔG profile may be learned by a prediction model according to various embodiments;

FIG. 18A shows waypoints of a free energy ΔG profile and in silico behavior of a reaction compared to expected behavior of the reaction prior to fitting the curves and learning the waypoints according to various embodiments;

FIG. 18B shows waypoints of a free energy ΔG profile and in silico behavior of a reaction compared to expected behavior of the reaction after fitting the curves and learning the waypoints according to various embodiments;

FIG. 19 illustrates an example of a multi-substrate enzymatic reaction according to various embodiments;

FIG. 20 shows an example of a Michaelis-Menten rate equation for a multi-substrate reaction according to various embodiments;

FIG. 21 shows a free energy landscape for a multi-substrate enzymatic reaction according to various embodiments;

FIG. 22 shows an example of a free energy profile according to various embodiments;

FIG. 23 illustrates a process according to various embodiments;

FIG. 24 shows an example enzymatic reaction between a substrate S and an enzyme E to form a product P with the associate rate constants according to various embodiments;

FIG. 25 shows a plot of the catalytic rate constant k_{cat} against the Michaelis constant Kₘ for an enzymatic reaction according to various embodiments; and

FIG. 26 shows an example computing device suitable for modeling in silico the kinetics of systems of connected biochemical reactions according to various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### I. Introduction

The present disclosure describes a general and scalable techniques for modeling in silica the kinetics of systems of connected biochemical reactions. General means that core “building block” components can be assembled to represent the detailed mechanism of most biochemical processes such as enzymatic reactions. Scalable means that these components can be systematically translated into standard mathematical constructs regardless of the size of the biological system.

A fundamental challenge to the modeling of a biological system such as a whole cell is integrating the kinematic behavior of individual enzymes and enzymatic reactions to the cellular level over several spatial and temporal scales (i.e., predict the rate at which each reaction in a system is running in parallel given any condition). This is challenging because it requires accurate parameter values and scalable methods for simulating large models. To overcome this fundamental challenge, biochemical systems have been modeled using a simplified irreversible reaction shown in Equation (1) known as the Michaelis-Menten equation, which only requires three kinetic parameters.

$$E + S {{⇌}^{{k}_{1}}}_{{k}_{- 1}} ES {⇀}^{{k}_{2}} E + P \qquad Equation ( 1 )$$

where S, E, ES, and P denote the substrate, enzyme, enzyme-substrate complex, and product, respectively. The full mass action description of this reaction requires the three kinetic parameters: k₁, k₋₁ and k₂, which are the forward association, dissociation and catalytic rate parameters, respectively. Each of the intermediate reactions in Equation 1 is considered a microscopic step or microstep of the overall reaction. An enzymatic reaction include a plurality of microsteps (e.g., binding of an enzyme to a substrate, transitioning of an enzyme:substrate complex to an enzyme:product substrate, or dissociation of a product from an enzyme:product complex).

Although the Michaelis-Menten equation provides an approximation of the single enzyme action, in vitro studies of single molecule enzyme kinetics have shown that this approximation is only sufficient in experiments where there is only one substrate/product and no initial product at the start of the system. However, there is a desire to model systems with in vivo conditions that provide a product at the start and/or multiple substrate/products. To overcome these limitations, modifications to the Michaelis-Menten equation have led to other enzyme kinetic equations and models such as a reversible Michaelis-Menten model and the Tzafriri total quasi-steady state assumption (tQSSA) model. Whilst the reversible Michaelis-Menten model is more widely used, it is strictly accurate at low enzyme concentrations. Since this may not be true under many in vivo conditions, unrealistic conclusions may be drawn from biological system models using the reversible Michaelis-Menten model. The tQSSA model is not subject to the same limitation as the reversible Michaelis-Menten model, but the tQSSA model has a more complex mathematical form that requires reanalysis for each distinct network to which it is applied. Currently, system and process modelers must choose between complex enzyme kinetics models with high parameter dimensionality, or simpler enzyme kinetics models at the cost of accuracy.

Enzymatic reactions with an enzyme binding with multiple substrates increase the number of rate constants, concentrations, and equations in modeling. Multiple substrates, each binding individually to an enzyme, may increase the number of microsteps to consider in an overall enzymatic reaction. As explained above, the introduction of two substrates binding with an enzyme may result in eight rate equations and eight rate constants. Each rate equation involves the concentration of the enzyme and at least one of the two substrates. Conventional methods of modeling enzymatic reactions may involve solving a system of these rate equations, each with its own rate constant. With conventional methods of modeling an enzymatic reaction, accurate or approximate values for rate constants are desired. Obtaining values for these rate constants may be difficult experimentally. The rates in forming enzyme and single substrate complexes and then in forming enzyme and double substrate complexes may be hard to measure as the complexes themselves may not be long lasting.

Moreover, simplifying assumptions that may be realistic for a single-substrate enzymatic reaction may not be realistic for a multi-substrate enzymatic reaction. Typical simplifying assumptions for a single-substrate enzymatic reaction include assuming that the rate constant for an enzyme-substrate complex ES dissociating is much larger than the rate constant of ES forming an enzyme-product complex EP. With multiple substrates S₁ and S₂, an analogous assumption may be that ES₁S₂ dissociating is much larger than forming EP₁P₂, where P₁ and P₂ are products. At the same time, enzyme-substrate complexes ES₁ and ES₂ are formed and one may wish to assume that the dissociation of ES₁ and ES₂ are faster than forming ES₁S₂. The observed behavior may not support these simplifying assumptions, and such observations are more difficult for multi-substrate enzymatic reactions. These common simplifying assumptions essentially zero out a rate constant and a reaction. The consequence of the simplifying assumption being incorrect may be that the overall enzymatic reaction itself, or a microstep of the overall enzymatic reaction, may not be consistent with observed behavior for the specific reaction or enzymatic reactions in general. Simplifying assumptions may result in other rate constants having values higher than theoretical or realistic maxima for rate constants.

Some embodiments of the present invention may avoid the issues with increased number of unknowns in conventional modeling approaches. The approaches described herein on using free energy profiles to model a multi-substrate enzymatic reaction. The free energy change (e.g., ΔG where G is the Gibbs free energy) for the entire enzymatic reaction may be known. The free energy at intermediate steps within the entire enzymatic reaction may be approximated. The free energy of these intermediate steps may be adjusted until the modeled catalytic rate constant k_{cat} and Michaelis constant Kₘ are similar to the experimentally observed k_{cat} and Kₘ. The rate constants for the microsteps can be calculated from the free energies. As a result, unknown rate constants introduced with multiple substrates in an enzymatic reaction may not significantly impair the modeling of the multi-substrate enzymatic reaction.

The approaches described herein may use as inputs a net change in free energy for the overall enzymatic reaction, a catalytic rate constant k_{cat}, and a Michaelis constant Kₘ to model a multi-substrate enzymatic reaction. The free energy change, k_{cat}, and Kₘ may be obtained theoretically or experimentally. The approaches may include creating a free energy profile rather than using a system of rate equations with rate constants to model the behavior. As a result, the approaches described herein may be more tolerant of unknown values for rate constants and may be more accurate than conventional methods using rate equations.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent. As used herein, when an action is “based on” something, this means the action is based at least in part on at least a part of the something.

### II. Interaction System and Biological System Modeling Techniques

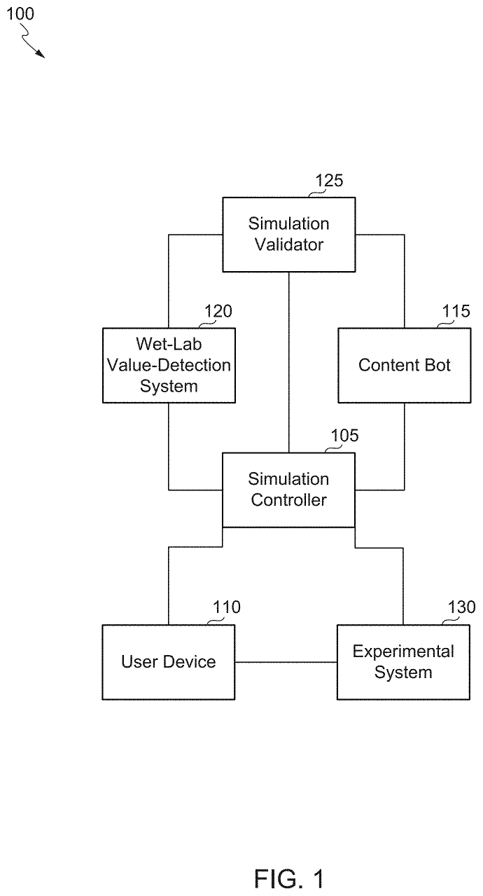

FIG. 1 shows an interaction system 100 for configuring instances or versions of a model and using a simulation to facilitate subsequent experiment configurations (e.g., simulation of a biological system's response to a new demand) according to various embodiments. Each instance of the models may have a combination of modules, perturbations (such as knockouts), and may be built using a particular set of experimental data. In order to facilitate the configuring of a model (e.g., a biological system) and simulate an outcome of the model, the interaction system 100 can include one or more components, each of which can include (for example) one or more servers, one or more computers and/or one or more mobile devices. In some instances, two or more of the components can be included in a same server, same server system, same computer, etc. Interaction system 100 can include one or more networks (e.g., a wired network, a wireless network, the Internet, a local area network, a wide area network, a short-range network, etc.), such that each component in the interaction system 100 can communicate with one or more other components in the interaction system 100.

Interaction system 100 can include a simulation controller 105 that defines, generates, updates and/or executes each of one or more simulations. A simulation can be configured to simulate dynamic progression through states, a time-evolved state of a model of a biological system and/or a steady state based on an iterative module-based assessment. It will be appreciated that identifying a steady-state and/or balanced solution for a module at a given time step need not indicate that a steady-state and/or balanced solution has been, can be or will be identified for the model in general (e.g., as metabolites produced and/or consumed at one module may further be produced and/or consumed at another module that need not be configured for balancing fluxes).

A given model can be used to generate and run any number of simulations. Differing initial conditions and/or differing automatically generated values in stochastic portions of the simulation (e.g., generated using a pseudo-random number generation technique, a stochastic pull from a distribution, etc.) can result in different output results of different simulations. The biological system model can be made up of one or more modules, and during a simulation run, each module is run independently and passes results back up to the biological system model level. More specifically, the biological system (e.g., a whole cell) may be modeled in accordance with a coordinated operation of multiple modules that represent structure(s) and/or function(s) of the biological system. Each module may be defined to execute independently, except that a shared set of state values (e.g., a state vector) maintained at the biological system model level may be used and accessed by multiple modules at each time point.

In some instances, each module of the biological system is configured to advance across iterations (e.g., time points) using one or more physiological and/or physics-based models (e.g., flux balance analysis (FBA), template synthesis, bulk-mass flow analysis, constant non-specific degradation, empirical analysis, etc.). The module-specific iteration processing can further be based on one or more module-specific state values (as determined based on an initial definition for an initial iteration processing or a result of a previous iteration processing for a subsequent iteration processing). The module-specific iteration processing can further be based on one or more parameters defined for the module that are fixed and/or static across iterations across iterations.

Simulation controller 105 can generate simulation configurations using one or more inputs received from a user device 110. For example, simulation controller 105 may generate an interface (or may at least partly define specifications for an interface) that is to be availed and/or transmitted to user device 110 and to include input fields configured to receive inputs that correspond to a selection of (for example) one or more modules to be used for a given biological system model, a model type to be used for each of the one or more modules, one or more parameters that are to be effected by a given module's model and used during execution, and/or one or more initial state-value definitions that are to be used by a given module's model and used during execution. In some instances, the interface identifies a default value for each of one, more or all parameters of the model and for each of one, more or all of the initial-state values of the model and is configured to receive a modification to a subset or all of the parameters and/or initial-state values for which a default value was identified. In some instances, modifying a default initial-state value and/or parameter can correspond to a perturbation of performance of a corresponding module and/or the biological system.

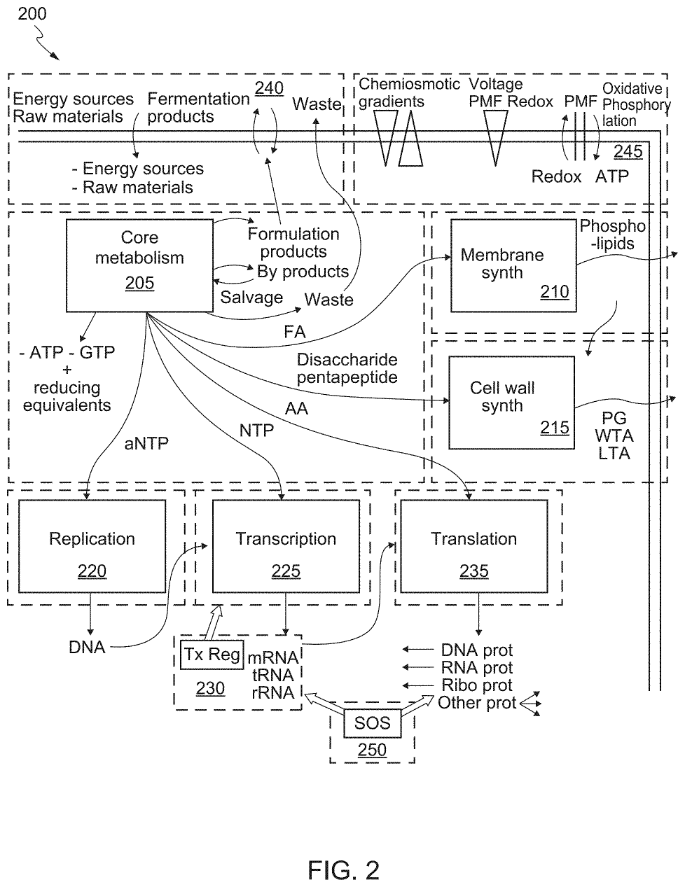

As another example, the interface may further or alternatively be configured to receive an input that corresponds to a selection of one or more default modules and a selection of a model type to be used for each of one or more modules. For example, the interface may include one or more modules (as shown in FIG. 2) representing distinct biological functions in a biological system model, and for each module: a name of the module, a default model type for the module and an option configured to receive a selection of another model type for the module (e.g., that identifies one or more other model types that can be selected for the module).

Default structure of a simulation (e.g., corresponding to default modules, default parameters, default initial-state values and/or default model selections) can be determined based on detected internal or external content and/or based on lab results (e.g., results from physical experiments). The content can include (for example) online, remote and/or local content that is collected by a content bot 115. Content bot 115 can (for example) include a crawler that performs a focused crawling and/or focused browsing (for example) the Internet, a part of the Internet, one or more pre-identified websites, a remote (e.g., cloud-based) storage system, a part of a remote storage system, a local storage system and/or a part of a local storage system. The crawling can be performed in accordance with one or more crawling policies and/or one or more queries that corresponds to one or more modules and/or models (e.g., where each query includes a variable name, representation or description and/or a cellular-function name, representation or description).

The lab results can be received from a wet-lab value detection system 120, which can be configured to trigger performance of one or more investigations (e.g., physical experiments) to detect and/or measure data corresponding to an initial-state value and/or data corresponding to a characteristic or parameter of a biological system. Wet-lab value-detection system 120 can transmit one or more results of the investigation(s) back to simulation controller 105, which may thereafter determine and/or define a default initial-state value or parameter or a possible modification thereof based on the result(s).

Interaction system 100 further includes a simulation validator 125, which can be configured to validate performance of a simulation. The validation may be performed based on pre-identified indications as to how a biological system functions normally and/or given one or more perturbations. Such indications can be defined based on content collected from content bot 115 and/or results from wet-lab value-detection system 120. The data used to validate the simulation may include (for example) one or more balanced values, one or more values indicative of cell dynamics, one or more steady-state values, one or more intermediate values and/or one or more time-course statistics. Simulation validator 125 may return a performance result that includes (for example) a number, category, cluster or binary indicator to simulation controller 105. Simulation controller 105 may use the result to determine (for example) whether a given simulation configuration is suitable for use (e.g., in which case it may be selectable in an interface).

After a simulation is configured with definitions and/or selections of modules, module-specific models, parameters and/or initial-state values, simulation controller 105 can execute the simulation (e.g., in response to receiving an instruction from user device 110 to execute the simulation). The simulation execution can produce one or more simulation results, which may include (for example) one or more balanced values, kinetic values, etc. For example, the simulation can identify a solution for a set of reaction-corresponding stoichiometric equations using linear algebra, such that production and consumption of metabolites represented in the equations is balanced. Notably, this balance may be specific to a given module and need not be achieved for all metabolites produced or consumed by reactions for a given module (e.g., as a non-zero net production or consumption of one or more boundary metabolites may be pre-defined and/or a target result for a module). Simulation controller 105 can transmit the results (e.g., via an interface) to user device 110.

In some instances, the results can be used to trigger and/or define a subsequent experiment. For example, simulation controller 105 may determine whether a given predefined condition is satisfied based on the results and, if so, may transmit simulation-specific data (e.g., indicating one or more initial-state values, parameters, mutations corresponding to simulation definitions, etc.) to an experimental system 130. The transmission may be indicative of and/or include an instruction to perform an experiment that corresponds to the simulation.

As another example, upon receiving simulation results from simulation controller 105, user device 110 can present an interface that includes some or all of the results and an input component configured to receive input corresponding to an instruction to perform an experiment that corresponds to the simulation. Upon receiving a selection at the input component, user device 110 may transmit data corresponding to the simulation to experimental system 130. After performing a requested experiment, experimental system 130 may return one or more results to simulation controller 105 and/or user device 110.

FIG. 2 shows an illustrative representation of given biological system model 200. The overall modeling strategy includes partitioning the biological system model 200 into modules that can be modeled separately, using a methodology and level of detail appropriate to and/or selected for each module. The partitioning and level of detail for each module can be selected based on (for example) the experiments or simulations that are to be run by the model (e.g., the questions trying to be solved by the model). The selection may be made by the modeler and/or computing system (e.g., the interaction system 100 described with respect to FIG. 1). For example, a user working through an interface of an integrated development environment, a script, and/or an automated system may be implemented to select one or more modules and select a model type to be used for each of one or more modules to ultimately generate the biological system model 200. Additionally or alternatively, the partitioning can be customized and depend on an assessment of the biological functions defined for the initial high-level data set. For example, a separate module may be defined to represent each of the following biological functions: core metabolism 205, membrane synthesis 210, cell-wall synthesis 215, DNA replication 220, transcription 225, transcription regulation 230, translation 235, RNA salvage (not shown), protein and RNA maturation, protein salvage (not shown), transmembrane transport 240 (including electron chain, oxidative phosphorylation, redox, and pH interconversion activity 245), signal transduction (not shown), stress response and growth rate regulation 250, cell division, chemotaxis (not shown), and cell-cell signaling (not shown).

Biological system model 200 can include at least one module that handles core metabolism 205. One possible core metabolic module uses an FBA model, which takes its general shape from standalone FBA, but includes modifications that account for interactions of the core metabolic module with other modules. Each of one, more or all other modules may have their own production and consumption of some of the same molecules within the FBA network, as described in further detail herein. However, as should be understood to those of ordinary skill in the art, an FBA model does not have to be incorporated into the overall biological system model 200 in order for every simulation to work. Instead, various types of models can be used for the modules (e.g., core metabolism 205, membrane synthesis 210, cell-wall synthesis 215, etc.) so long as the type of models can be configured to read values from the state vector and return a list of changes that should be made to the state vector.

For one exemplary instantiation of biological system model 200, core metabolism 205, membrane synthesis 210, and cell-wall synthesis 215 may be encompassed as a single FBA problem, whereas DNA replication 220, transcription 225, transcription regulation 230, and translation 235 may be isolated from the rest of the metabolic network. Meanwhile, transcription 225 and translation 235 may use a template synthesis model, and DNA replication 220 may use a bulk mass-flow model. Transcription regulation 230 may be empirical and static. Optionally, RNA salvage may be modeled using constant non-specific degradation, polymerized DNA, RNA, and protein levels may be determined by the intrinsic rates of the processes that produce them, and the remainder of the components are provided as inputs or parameters of the model.

For another exemplary instantiation of biological system model 200, core metabolism 205 may be encompassed as a single FBA problem. The balance of internal metabolite pools and the supply of building blocks for other processes may be maintained by core metabolism 205. DNA replication 220, transcription 225, transcription regulation 230, and translation 235 may then be isolated from the rest of the metabolic network. Membrane biosynthesis 210 and cell-wall synthesis 215 may be modeled by substrate- and catalyst-driven kinetics. Import and export rates and all exchange with the environment may be driven by the kinetics of membrane transport. Transcription 225 and translation 235 may use a template synthesis model, and DNA replication 220 may use a bulk mass-flow model. Transcription regulation 230 may be empirical and static. Optionally, RNA salvage may be modeled using representations of constant non-specific degradation, while polymerized DNA, RNA, and protein levels may be determined by the intrinsic rates of the processes that produce them, and the remainder of the components for the biological system can be provided as inputs or parameters of the model.

For another exemplary instantiation of biological system model 200, core metabolism 205 may be encompassed as an FBA problem, whereas one or more of membrane synthesis 210, cell-wall synthesis 215, DNA replication 220, transcription 225, transcription regulation 230, and translation 235 can be isolated from the rest of the metabolic network. The balance of internal metabolite pools and the supply of building blocks for other processes may be maintained by core metabolism 205. Membrane biosynthesis 210 and cell-wall synthesis 215 may be modeled by substrate and catalyst driven kinetics. Import and export rates, and all exchange with the environment may be driven by the kinetics of membrane transport. Redox balance, pH, and chemiosmotic gradients may be maintained explicitly. DNA replication 220, transcription 225 and translation 235 may use models based on initiation, elongation, and termination, Transcription regulation 230 may be pattern driven. Stress response and growth rate regulation 250 may be modeled using feedback control mechanisms. Optionally, RNA salvage may be modeled using constant non-specific degradation, while polymerized DNA, RNA, and protein levels may be determined by the intrinsic rates of the processes that produce them, and the remainder of the components for the biological system can be provided as inputs or parameters of the model.

While the biological system model 200 has been described at some length and with some particularity with respect to several described modules, combinations of modules, and simulation techniques, it is not intended that the biological system model 200 be limited to any such particular module configuration or particular embodiment. Instead, it should be understood that the described embodiments are provided as examples of modules, combinations of modules, and simulation techniques, and the modules, combinations of modules, and simulation techniques are to be construed with the broadest sense to include variations of modules, combinations of modules, and simulation techniques listed above, as well as other modules, combinations of modules, and simulation techniques configurations that could be constructed using a methodology and level of detail appropriate to each module and the biological system model 200.

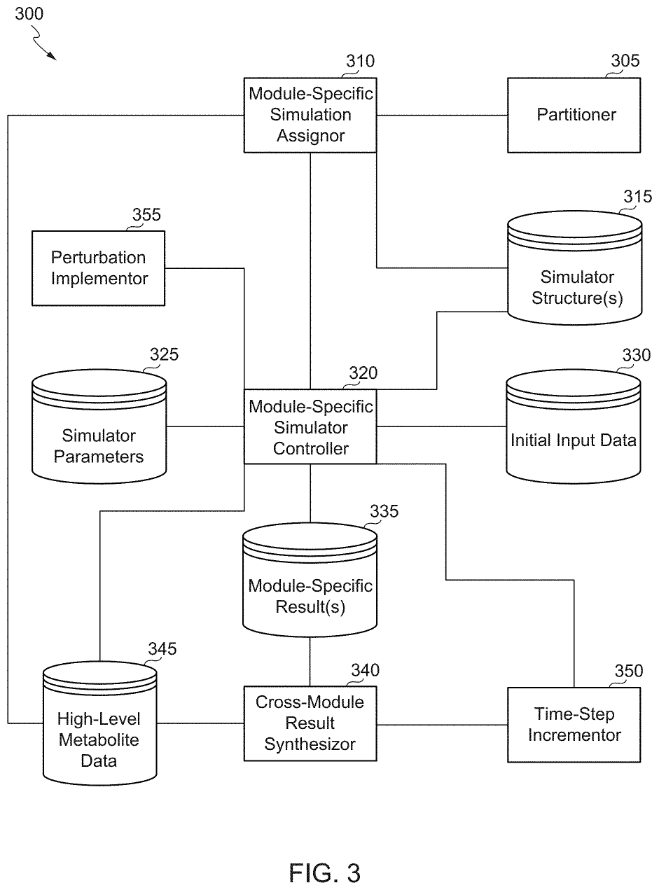

FIG. 3 shows a simulation controller 300 that dynamically integrates results generated by different types of models configured by an integrated development environment (e.g., the interaction system 100 described with respect to FIG. 1) to simulate higher-level states and reactions of a biological system model (e.g., biological system model 200 as described with respect to FIG. 2) according to various embodiments. A partitioner 305 that can identify one or more modules to potentially use for a simulation. In some instances, the modules are identified to correspond to distinct biological functions or physiological processes within a biological system model. Nonetheless, at least one module (e.g., a core module) may address in more detail or cover a larger set of biological functions (e.g., correspond to a core level of physiology across the biological system such as general metabolism of the biological system), whereas at least one other module (e.g., a non-core module) may address in less detail or cover a smaller set biological function (e.g., correspond to transcription and/or translation).

A module-specific simulation assignor 310 may assign, to each module, a simulation type. The simulation type can be selected from amongst one or more types that are associated with the module and/or corresponding physiological process. The one or more types may differ with regard to (for example) a degree of detail to which a physiological process is modeled and/or how the process is modeled. For example, the one or more types may include a simulation using a metabolism-integrated model (e.g., in which specific end products are added to an objective function of a metabolism-based model), substrate- and/or catalyst-drive model using kinetic parameters and reactions, and/or higher-order structure model. A structure for each simulation type (e.g., that indicates how the simulation is to be performed and/or program code) is included in a simulator structure data store 315. Simulator structure data store 315 can further store an association between each simulation type and one or more modules for which the simulation type is associated and is permitted for selection for use.

A module-specific simulator controller 320 can identify, for each module, one or more simulation parameters and an input data set. The simulation parameters may be retrieved from a local data store (e.g., a simulator parameters data store 325) or from a remote source. Each of one or more of the simulation parameters may have been identified based on (for example) user input, a data-fitting technique and/or remote content. The parameter(s), once selected, may be fixed across time-step iterations.

At an initial time step, the input data set can include one or more initial input values, which may be retrieved from a local data store (e.g., an initial input data store 330) or from a remote source. Each of one or more of the initial input values may have been identified based on (for example) user input, a data-fitting technique and/or remote content. With respect to each subsequent time step, the input data set can include (for example) one or more results from a previous iteration of the module and/or one or more high-level results (e.g., cumulative or integrated results) generated from a previous iteration of the multi-module simulation. For example, a module-specific results data store 335 may store each of one, more or all results generated by the assigned simulation for each of one, more or all past time steps, and at least one of the stored results associated with a preceding time step (e.g., most recent time step) can be retrieved.

Upon identifying the input data set and parameters, module-specific simulator controller 320 can run the simulation assigned to the module. Execution of module-specific simulations may be performed concurrently, in parallel and/or using different resources (e.g., different processors, different memory and/or different devices). Results of the simulation run can be stored in module-specific results data store 335.

After results have been generated for each module, a cross-module result synthesizor 340 can access the module-specific results (from one or more module-specific results data stores or direct data availing) and synthesize the results to update high-level data such as a state vector (e.g., stored in a high-level metabolite data store 345). For example, a set of results generated by different modules but relating to a same variable may be identified. The results may be integrated by (for example) summing variable changes as indicated across the results (e.g., potentially with the implementation of one or more caps pertaining to a summed change or to a value of a variable after the summed change is effected). In some instances, a hierarchy is used, such that a result from one module (if available or if another condition is met) is to be exclusively used and a result from another module is to otherwise be used.

Upon synthesizing the results, a time-step incrementor 350 can increment a time step to a next time step so long as the simulation has not completed. It may be determined that the simulation is complete when (for example) processing for a predefined number of time steps has been performed, a particular result is detected (e.g., indicating that a target cell growth has occurred or that a cell has died) or steady state has been reached (e.g., as indicated by values for one or more predefined types of results differing by less than a predefined threshold amount across time steps). When the time step is incremented, module-specific simulator controller 320 can, for each module, collect a new input data set and run the assigned simulation. When the simulation is complete, an output can be generated to include one or more module-specific results, some or all high-level data and/or processed versions thereof. For example, the output may include time-course data for each of one or more metabolites, growth of the biological system over a time period (e.g., as identified by a ratio of availability values of one or more particular metabolites at a final time step as compared to availability values at an initial time step) and/or a growth rate. The output can be transmitted to another device (e.g., to be presented using a browser or other application) and/or presented locally.

Multi-module simulation controller 300 can also include a perturbation implementor 355. Perturbation implementor 355 can facilitate presentation of an interface on a user device. The interface can identify various types of perturbations (e.g., mutations). Perturbation implementor 355 may facilitate the presentation by transmitting data (e.g., HTTP data) to a user device, such that the interface can be presented online. Perturbation implementor 355 can detect a selection that corresponds to a particular perturbation and can send an indication to module-specific simulator controller 320. Module-specific simulator controller 320 can use functional gene data to determine how the mutation affects one or more metabolites and/or one or more simulated processes. A structure of a simulator, one or more simulator parameters and/or one or more initial-input values may then be adjusted in accordance was the perturbation's effects.

Thus, multi-module simulation controller 300 can generate output that is indicative of how the perturbation affects (for example) physiological processes and/or growth of the biological system.

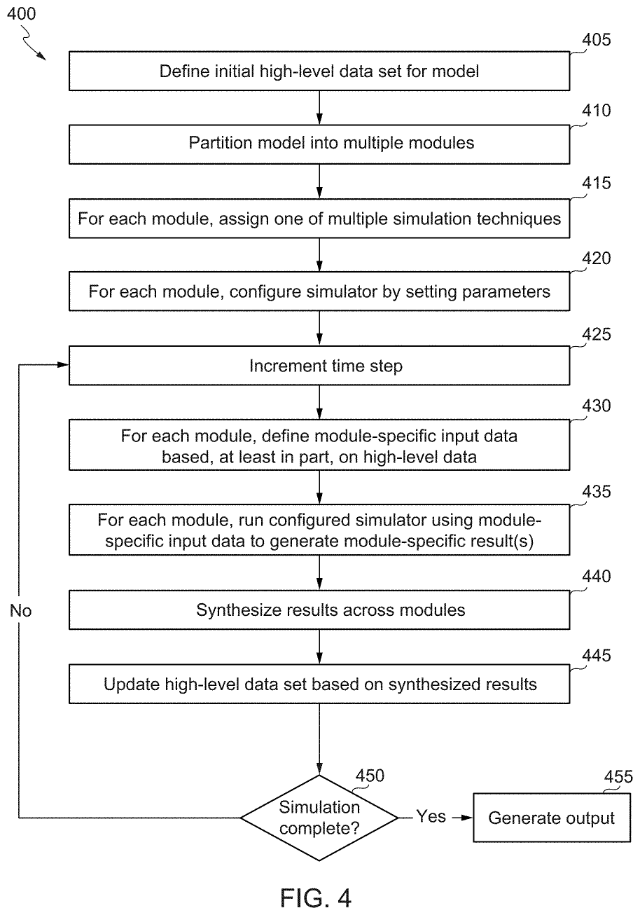

FIG. 4 shows a process 400 for dynamically synthesizing results generated by multiple simulators to simulate higher-level results according to various embodiments. In some embodiments, the processes depicted in process 400 are implemented by the interaction system 100 of FIG. 1, and discussed with respect to the simulation controller 300 of FIG. 4. Process 400 begins at block 405 at which an initial high-level data set is defined for a biological system model. The initial high-level data set can identify (for example) variables, which may be referred to as the state of the biological system model or the state of the simulation, and these variables may be structured as a data structure (e.g., a state vector) and updated throughout a simulation run. In some instances, the variables include an initial availability of each of a set of molecules such as metabolites. The initial availability may be defined based on (for example) a default value, user input, data extracted from content (e.g., online content, remote content or local content that pertains to the molecules), etc. In some instances, the initial availability is determined based on whether any perturbation was identified (e.g., via user input) for a given simulation. If a perturbation was identified, the initial availability may be determined based on a particular perturbation that was identified and by using (for example) a look-up table to determine for which molecule(s) the perturbation affects an availability value and characteristics of such effect.

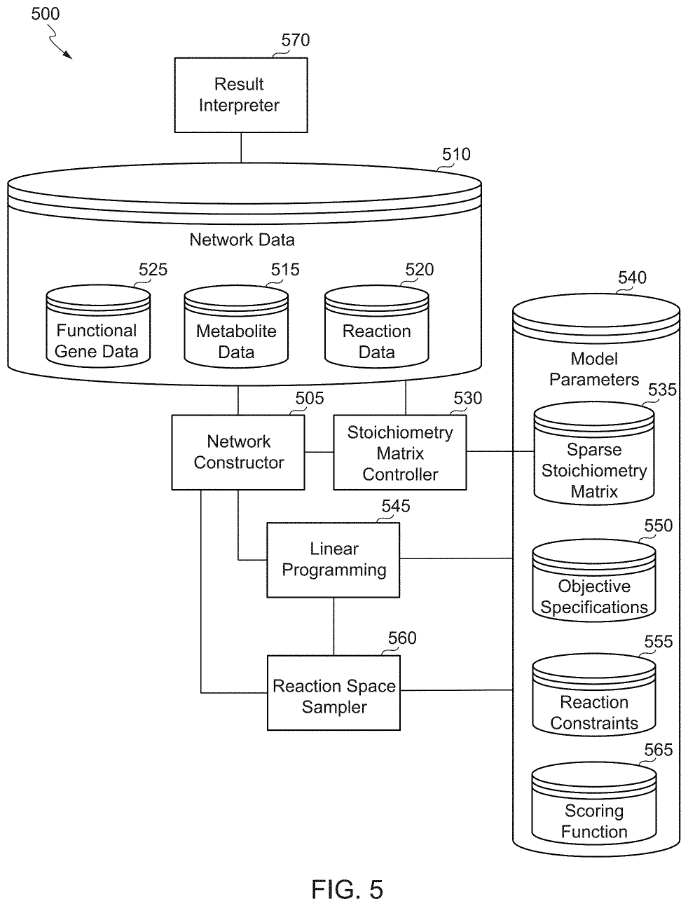

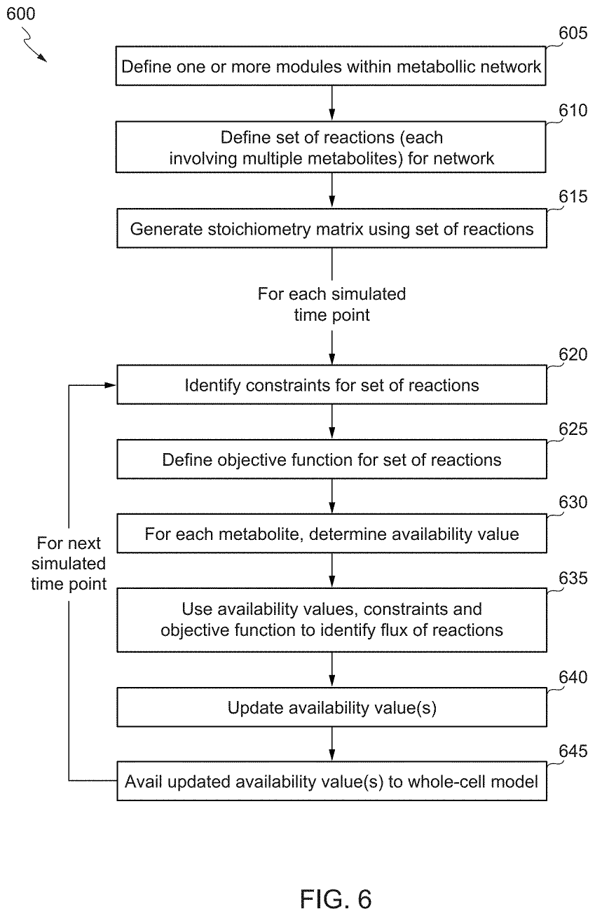

At block 410, a biological system model (e.g., a whole cell model) is partitioned into multiple modules. The partitioning can depend on metabolite dependencies and/or biological-functioning assessment. For example, a separate module may be defined to represent each of the following biological functions: core metabolism, membrane synthesis, cell-wall synthesis, DNA replication, transcription, transcription regulation, translation, RNA salvage, protein and RNA maturation, protein salvage, transmembrane transport (including electron chain, oxidative phosphorylation, redox, and pH interconversion activity), signal transduction, stress response and growth rate regulation (SOS), cell division, chemotaxis, and cell-cell signaling, as discussed in further detail with respect to FIG. 2. In some instances, two or more of these functions may be represented in a core module that models cell composition and growth using a single model. Particular cellular functioning need not be explicitly modeled and instead dynamics of end products of the particular cellular functioning may be modeled. For example, a core module may use a flux-based analysis or a simulation technique as described herein (e.g., in relation to FIG. 5 or FIG. 6).

In some instances, the partitioning may be performed based on user input and/or one or more default configurations. For example, an interface may be presented that identifies each potential separate module (e.g., an interface may be presented via simulation controller 105 as described with respect to FIG. 1). A default configuration may be to integrate the module into a core module (e.g., a core metabolism module) unless a contrary input is received or to perform a simulation using modeling specific to the module unless a contrary input is received. For example, an interface may be configured to receive one or more selections of modules that are to be excluded from a core module and to then integrate each other module into the core module.

At block 415, for each module, one or more simulation techniques are assigned to the module. A simulation technique may include a model type. In some instances, a simulation technique that is assigned to a core module includes a flux-based analysis or other simulation technique, as described herein. In some instances, a simulation technique includes a mechanistic model, a kinetic model, a partial kinetic model, a substrate- and/or catalyst-driven model, and/or a structural model. The simulation technique may be assigned based on (for example) user input and/or one or more predefined default selections. For example, for each non-core module, a default selection may be predefined that represents particular functioning of the module, and for each core module, a default selection may be predefined that simulates dynamics of metabolites across a simulated time period. An interface may identify, for each module, the default selection along with one or more other simulation techniques that are associated with the module (e.g., with the association(s) being based on stored data and/or a predefined configuration). User input may then indicate that an alternative simulation technique is to be used for one or more modules.

At block 420, for each module, a simulator is configured by setting parameters and variables. The parameters (e.g., numeric values) may correspond to inputs to be used in the simulation technique assigned to the module and that are not changed across time steps of the simulation. The particular parameters may be determined based on (for example) stored data, content, a communication from another system and/or user input. The one or more module-specific or cross-module variables (e.g., identifying an initial availability of one or more metabolites) may correspond to inputs to be used in the simulation technique assigned to the module and may be changed across time steps of the simulation. For example, a parameter may be determined for a simulator that sets a minimum viable pH in the cytoplasm (below which the cell dies), and a variable may be identified that describes a current pH in the cytoplasm. The variable (current pH) might change throughout the simulation; however, the parameter (the minimum possible pH) would not change and remains fixed. An initial value of the pH variable may be identified, e.g., the value at the start of the simulation may be set in block 405 or if it is module specific then it may be set in block 420, and like the minimum pH parameter this would be used as an input into the simulation. The values of variables and parameters are both inputs, but the distinction is that variables can change from their initial values, and parameters are fixed throughout the simulation run.

At block 425, a time step is incremented, which can initially begin a given simulation. At block 430, for each module, module-specific input data is defined at least in part on the high-level data. More specifically, a high-level data structure may identify, for each of a set of molecules (e.g., metabolites), an availability value. Each availability value may initially be set to an initial availability value, which may thereafter be updated based on processing results from each module that relates to the molecule. For a given module, at each time step, a current availability value can be retrieved from the data structure for each molecule that pertains to the simulation technique assigned to the module. The module-specific input data may further include one or more lower-level values that are independent from processing of any other module. For example, one or more variables may only pertain to processing of a given module, such that the module-specific input data may further include an initial value or past output value that particularly and exclusively relates to the module.

At block 435, for each module, the configured simulator assigned to the module is run using the module-specific input data to generate one or more module-specific results. The one or more module-specific results may include (for example) one or more updated molecule availability values and/or a change in one or more availability values relative to corresponding values in the input data.

At block 440, results can be synthesized across modules. The synthesis may include summing differences across modules. For example, if a first module's results indicate that an availability of a given molecule is to be increased by 5 units and a second module's results indicate that an availability of the given metabolite is to be decreased by 3 units, a net change may be calculated as being an increase in 2 units. The net change can then be added to a corresponding availability value for the molecule that was used for the processing associated with the current time step and returned as a list of changes that should be made to the state vector. One or more limits may be applied to a change (e.g., to disallow changes across time steps that exceed a predefined threshold) and/or to a value (e.g., to disallow negative availability values and instead set the value to zero).

At block 445, the high-level data set is updated based on the synthesized results. The update can include adding data to a data structure such as a state vector from which one or more modules retrieve high-level data. The added data can include the synthesized results in association with an identifier of a current time step. Thus, the data structure can retain data indicating how an availability of a metabolite changed over time steps. It will be appreciated that alternatively the update can include replacing current high-level data with the synthesized data.

At block 450, it is determined whether the simulation is complete. The determination may be based on a number of time steps assessed, a degree to which data (e.g., high-level data) is changing across time steps, a determination as to whether a steady state has been reached, whether one or more simulated biological events (e.g., cell division or cell death) have been detected, etc. If the simulation is not complete, process 400 returns to block 425.

If the simulation is complete, process 400 continues to block 455, at which an output is generated. The output may include some or all of the high-level data and/or some or all of the module-specific results. For example, the output may include final availability values that correspond to a set of metabolites and/or a time course that indicates a change in the availability of each of one or more metabolites over the simulated time period. The output may be presented at a local device and/or transmitted to another device (e.g., for presentation).

FIG. 5 shows a module-specific simulation controller 500 to simulate states and reactions of modules configured by an integrated development environment (e.g., the interaction system 100 described with respect to FIG. 1) according to various embodiments. A network constructor 505 can be configured to use a model to simulate actions performed by a module of a biological system model (e.g., biological system model 200 as described with respect to FIG. 2). In some instances, the model is flux balance analysis, and/or the model is configured to solve for updated state values based on a set of equations that represent concentration changes in the network (e.g., a metabolic network). As should be understood to those of ordinary skill in the art, a biological system model such as a whole cell model does not have to include an FBA module. For example, from the framework described herein, biological processes such as core metabolism may be modeled that is completely different from FBA. In such an instance, part or all of the description and drawings pertaining to FIGS. 5 and 6 that is specific to FBA (e.g., objective functions, constraints, and linear programming) may not be relevant to that particular instantiation of the model or to simulations run with that model. However, many of the components and techniques described with respect to FIGS. 5 and 6 could be applied to simulate states and reactions of modules implemented by other models. For example, any module can read values from the state vector and return an indication of one or more changes that should be made to the state vector. The FBA module (if it's even present in a particular instantiation of the model) may read and return more values than any other model, but a module modeled with FBA need not be handled by the simulation controller 300 any differently from other modules and/or models described herein.

Network constructor 505 can access a set of network data (e.g., parameters and variables) stored in a network data store 510 to define the model. Metabolite data 515 can identify each metabolite of a metabolome. As used herein, a “metabolite” is any substance that is a product of metabolic action or that is involved in a metabolic process including (for example) each compound input into a metabolic reaction, each compound produced by a metabolic reaction, each enzyme associated with a metabolic reaction, and each cofactor associated with a metabolic reaction. The metabolite data 515 may include for each metabolite (for example) one or more of the following: the name of the metabolite, a description, neutral formula, charged formula, charge, spatial compartment of the biological system and/or module of the model, and identifier such as PubChem ID. Further, metabolite data 515 can identify an initial state value (e.g., an initial concentration and/or number of discrete instances) for each metabolite.

Reaction data 520 can identify each reaction (e.g., each metabolic reaction) associated with the model. For example, a reaction can indicate that one or more first metabolites is transformed into one or more second metabolites. The reaction need not identify one-to-one relationships. For example, multiple metabolites may be defined as reaction inputs and/or multiple metabolites may be defined as reaction outputs. The reaction data 520 may include for each reaction (for example) one or more of the following: the name of the reaction, a reaction description, the reaction formula, a gene-reaction association, genes, proteins, spatial compartment of the biological system and/or module of the model, and reaction direction. Further, the reaction data 520 can identify, for each metabolite of the reaction, a quantity of the metabolite, which may reflect the relative input-output quantities of the involved metabolites. For example, a reaction may indicate that two first metabolites and one second metabolite are input into a reaction and that two third metabolites are outputs of the reaction. The reaction data 520 can further identify an enzyme and/or cofactor that is required for the reaction to occur.

Functional gene data 525 can identify genes and relationships between genes, proteins, and reactions, which combined provide a biochemically, genetically, and genomically structured knowledge base or matrix. Functional gene data 525 may include (for example) one or more of the following: chromosome sequence data, the location, length, direction and essentiality of each gene, genomic sequence data, the organization and promoter of transcription units, expression and degradation rate of each RNA transcript, the specific folding and maturation pathway of RNA and protein species, the subunit composition of each macromolecular complex, and the binding sites and footprint of DNA-binding proteins. Network constructor 505 can use functional gene data and the availability of proteins encoded by those genes to update reaction constraints. One exemplary technique by which genomic data can be associated with reaction data is evaluating Gene-Protein-Reaction expressions (GPR), which associate reactions with specific genes that triggered the formation of one or more specific proteins. Typically a GPR takes the form (Gene A AND Gene B) to indicate that the products of genes A and B are protein sub-units that assemble to form a complete protein and therefore the absence of either would result in deletion of the reaction. On the other hand, if the GPR is (Gene A OR Gene B) it implies that the products of genes A and B are isozymes (i.e., each of two or more enzymes with identical function but different structure) and therefore absence of one may not result in deletion of the reaction. Therefore, it is possible to evaluate the effect of single or multiple gene deletions by evaluation of the GPR as a Boolean expression. If the GPR evaluates to false, the reaction is constrained to zero in the model.

A stoichiometry matrix controller 530 can use reaction data 520 to generate a stoichiometry matrix 535. Along a first dimension of the matrix, different compounds (e.g., different metabolites) are represented. Along a second dimension of the matrix, different reactions are represented. Thus, a given cell within the matrix relates to a particular compound and a particular reaction. A value of that cell is set to 0 if the compound is not involved in the reaction, a postive value if the compound is one produced by the reaction and a negative value if the compound is one consumed by the reaction. The value itself corresponds to a cofficient of the reaction indicating a quantity of the compound that is produced or consumed relative to other compound consumption or production involved in the reaction.

Because frequently relatively few reactions correspond to a given compound, stoichiometry matrix 535 can be a sparse stoichiometry matrix. Stoichiometry matrix 535 can be part of a set of model parameters (stored in a model-parameter data store 540) used to execute a module.

One or more modules may be configured to use linear programming 545 to identify a set of compound quantities that correspond to balancing fluxes identified in reactions represented in stoichiometry matrix 535. Specifically, an equation can be defined whereby the product of stoichiometry matrix 535 and a vector representing a quantity for each of some of the compound quantities is set to zero. (It will be appreciated that the reactions may further include quantities for one or more boundary metabolites, for which production and consumption need not be balanced.) There are frequently multiple solutions to this problem. Therefore, an objective function is defined, and a particular solution that corresponds to a maximum or minimum objective function is selected as the solution. The objective function can be defined as the product between a transposed vector of objective weights and a vector representing the quantity for each compound. Notably, the transposed vector may have a length that is equal to the first dimension of stoichiometry matrix 535, given that multiple reactions may relate to a same compound.

The objective weights may be determined based on objective specifications 550, which may (for example) identify one or more reaction-produced compounds that are to be maximized. For example, the objective weights can be of particular proportions of compounds that correspond to biomass, such that producing compounds having those proportions corresponds to supporting growth of the biological system.

Each reaction may (but need not) be associated with one or more of a set of reaction constraints 555. A reaction constraint may (for example) constrain a flux through the reaction and/or enforce limits on the quantity of one or more compounds consumed by the reaction and/or one or more compounds produced by the reaction.

In some instances, linear programming 545 uses stoichiometry matrix 535 and reaction constraints 555 to identify multiple solutions, each complying with the constraints. When multiple solutions are identified, objective specifications 550 can be used to select from amongst the potential solutions. However, in some instances, no solution is identified that complies with stoichiometry matrix 535 and reaction constraints 555 and/or the only solution that complies with the matrix and constraints is not to proceed with any reaction.

A solution can include one in which, for each of a set of metabolites, a consumption of the metabolite is equal to a production of the metabolite. That is not to say that this balance must be achieved for each metabolite, as a set of reactions involve one or more “boundary metabolites” for which this balance is not achieved. For example, glucose can be consumed at a given rate, and/or acetate can be produced at a given rate.

Reaction data 520 may further identify an objective function that identifies a target product (e.g., representing cell growth rate) that is to be maximized. The objective function can identify particular ratios of multiple reactant metabolites that must be available to produce the product. Strictly enforcing the objective function may result in simulating no growth if a single metabolite is not produced. An alternative approach is to define one or more objective functions configured such that production of each of multiple target reactant metabolites that relate to the target product is to be maximized. A higher level whole-cell model can evaluate the production of multiple target reactant metabolites to determine whether to and/or an extent to which to simulate growth. For example, depending on which target reactant metabolite(s) are not produced, the whole-cell model may nonetheless simulate cell growth, simulate cell growth at a reduced rate, simulate no growth, simulate unhealthy or impaired growth or simulate cell death.

For example, a reaction space can be defined based on stoichiometry matrix 535 and reaction constraints 555. The space may have as many dimensions as there are reactions. Each dimension can be restricted to include only integer values that extend along a range constrained by any applicable constraint in reaction constraints 555. A reaction space sampler 560 can then determine, for each of some or all of the points within the reaction space, a cumulative quantity of each metabolite that would be produced based on the associated reactions. Reaction space sampler 560 can compare these quantities to those in the objective vector (e.g., by determining an extent to which proportions of compounds are consistent).

In these instances, a scoring function 565 can indicate how to score each comparison. For example if proportions of each of two potential solutions differ from the objective proportions by 2, but one potential solution differs by 2 for a single compound and another by 1 for each of two compounds, scoring function 565 can be configured to differentially score these instances. For example, different weights may be applied to different compounds, such that differences that affect a first compound are more heavily penalized than differences that affect a second compound. As another example, scoring function 565 may indicate whether a score is to be calculated by (for example) summing all compound-specific (e.g., weighted) differences, summing an absolute value of all compound-specific (e.g., weighted) differences, summing a square of all compound-specific (e.g., weighted) differences, etc. Reaction space sampler 560 can then identify a solution as corresponding to reaction coefficients that are associated with a highest score across the reaction space.

Network constructor 505 can receive results from each of linear programming 545 and/or reaction space sampler 560. In some instances, linear programming 545 can further avail its results to reaction space sampler 560. When a balanced solution is identified by linear programming 545, reaction space sampler 560 need not sample the reaction space and need not avail reaction-space results to network constructor 505.

Network constructor 505 can identify a solution as corresponding to one identified by linear programming 545 when a balanced solution is identified and as a highest-score potential solution identified by reaction space sampler 560 otherwise. The solution can then indicate the compounds produced by and consumed by the reactions performed in accordance with the solution-indicated flux. Network constructor 505 can update metabolite data 515 based on this production and consumption.

In some instances, a solution is identified for each of a set of time points rather than only identifying one final solution. The iterative time-based approach may be useful when module-specific simulation controller 500 is but one of a set of simulation controllers and metabolite data 515 is influenced by the performance of other modules. For example, metabolite data 515 may be shared across modules or may be defined to be a copy of at least part of a cross-module metabolite data set at each time point. The updates to the metabolites performed by network constructor 505 may then be one of multiple updates. For example, an update by network constructor 505 may indicate that a quantity of a specific metabolite is to increase by four, while a result from another module indicates that a quantity of the specific metabolite is to decrease by two. Then the metabolite may change by a net of +2 for the next time iteration.

A results interpreter 570 can generate one or more results based on the updated metabolite data 515. For example, a result may characterize a degree of growth between an initial state and a steady state or final time point. The degree of growth may be determined based on a ratio between values of one or more metabolites at a current or final time point relative to corresponding values at an initial (or previous) time point. The one or more metabolites may correspond to (for example) those identified in an objective function as corresponding to biomass growth. As another example, a result may characterize a time course of growth. For example, a result may identify a time required for metabolite changes that correspond to a representation of a double in growth or a time constant determined based on a fit to values of one or more time series of metabolite values. The result(s) may be output (e.g., locally presented or transmitted to a remote device, such as a user device). The output can facilitate a presentation of an interface that indicates one or more simulation characteristics (e.g., one or more default values in terms of initial-state values or reaction data and/or one or more effected perturbations).

Operation of module-specific simulation controller 500 can be influenced by particular simulated perturbations of the whole cell. For example, each perturbation may correspond to a particular type of genetic mutation. The perturbation may have been identified based on detecting user input (e.g., a selection and/or text input received via an interface) that defines the perturbation. One exemplary type of perturbation is a gene mutation. An effect of the perturbation may be determined based on functional gene data (e.g., to determine how an availability of one or more metabolites is affected). High-level metabolite data, simulator parameters and/or high-level constraints may then be accordingly set, constrained and/or defined based on the perturbation. This high-level perturbation can thus then influence operation of one or more lower level modules.

FIG. 6 shows a process 600 for using a simulator to generate metabolite time-course data according to various embodiments. In some embodiments, the processes depicted in process 600 are implemented by the interaction system 100 of FIG. 1, and discussed with respect to the module-specific simulation controller 500 of FIG. 5. Process 600 begins at block 605, at which a one or more modules within a metabolic network (e.g., of a biological system) are defined. The module(s) can be defined based on which parts of the network exhibit relative functional independence and/or correspond to substantial independence in terms of biological activity. In some instances, a default is to define each part of a cell as part of a core module unless a different module corresponding to particular types of actions and/or cell components is defined.

At block 610, a set of reactions is defined for the network. In some instances, the set of reactions are defined for the module (or each module) that corresponds to the default model type. The set of reactions can indicate how various molecules such as metabolites are consumed and produced through part of all of a life cycle of a biological system. Each reaction thus identifies one or more metabolites that are consumed, one or more metabolites that are produced and, for each consumed and produced metabolite, a coefficient (which may be set to equal one) indicating a relative amount that is consumed or produced. The reaction may further include an identification of one or more enzymes, one or my cofactors and/or one or more environmental characteristics that are required for the reaction to occur and/or that otherwise affects a probability of the reaction occurring or a property of the reaction. The reactions may be identified based on (for example) online or local digital content (e.g., from one or more scientific papers or databases) and/or results from one or more wet-lab experiments.

At block 615, a stoichiometry matrix is generated using the set of reactions. Each matrix cell within the matrix can correspond to a particular metabolite and a particular reaction. The value of the cell may reflect a coefficient of the particular metabolite within the particular reaction (as indicated in the reaction) and may be set to zero if it is not involved in the reaction. In some instances, metadata is further generated that indicates, for each of one or more reactions, any enzyme, co-factor and/or environmental condition required for the reaction to occur.

At block 620, one or more constraints are identified for the set of reactions. In some instances, identifying the constraints may include identifying values for one or more parameters. For example, for each of one or more or all of the set of reactions, a constraint may include a flux lower bound and/or a flux upper bound to limit a flux, a quantity of a consumed or produced metabolite, a kinetic constant, a rate of production or decay of a component such as RNA transcript, an enzyme concentration or activity, a compartment size, and/or a concentration of an external metabolite. The constraint(s) may be identified based on (for example) user input, online or local data, one or more communications from a wet-lab system, and/or learned from statistical inference.

At block 625, an objective function is defined for the set of reactions. The objective function may identify what is to be maximized and/or what is to be minimized while identifying a solution. The objective function may (for example) identify a metabolite that is produced by one or more reactions or a combination of metabolites that is produced by one or more reactions. The combination may identify proportions of the metabolites. However, the objective function can have a number of limitations and may fail to reflect supply and demand within the other modules. Thus, in some instances, a limited objective function can be constructed to include a set of target values for each molecule within the metabolic network. The target values can incorporate intrinsic-rate parameters, supply rates of molecules, the consumption rates of molecules, and the molecule concentrations into a measurement of target concentrations of the molecule given supply, demand, and an “on-hand” concentration of each molecule, which represents the concentration of a molecule immediately available to a reaction pathway. The target values may be calculated and incorporated into the objective function to produce the limited objective function. This may be in the form of calculating an absolute difference between the target value and the proportional flux contribution of each molecule. This may be in the form of scaling the proportional flux contribution of each molecule. This may be in the form of adding to the proportional flux contribution of each molecule. Any other mathematical modification of the proportional flux contribution of each molecule that adjusts this value by the target value may be used. The target values may be positive or negative. For purposes of unit conversion, so that target values can be included in the objective function and compared to the flux values, the target values may be constructed as rates.

At block 630, for each metabolite related to the set of reactions, an availability value is determined. For an initial value, the value may be identified based on (for example) user input, digital content and/or communication from another system. Subsequent values may be retrieved from a local or remote data object that maintains centralized availability values for the set of metabolites.

At block 635, the availability values, constraints and objective function are used to determine the flux of one, more or all of the set of reactions. The flux(es) may indicate a number of times that each of one, more or all of the reactions were performed in a simulation in accordance with the availability values, constraints and objective function. The flux(es) may be determined based on a flux-balance-analysis model. In some instances, the flux(es) may be determined based on a sampling of all or part of an input space representing different flux combinations and scoring each input-space using a scoring function.

At block 640, a centralized availability value of one or more metabolites is updated based on the determined flux(es). More specifically, for each metabolite, a cumulative change in the metabolite's availability may be identified based on the cumulative consumption and cumulative production of the metabolite across the flux-adjusted set of reactions. The centralized availability value of the metabolite can then be incremented and/or decremented accordingly.

In some instances, at least one the one or more modules defined at block 605 are to be associated with a model that does not depend on (for example) a stoichiometry matrix and/or flux based analysis and/or that is based on physiological modeling. One or more modules based on one or more different types of models can also, at each time point, identify a change in metabolite availability values, and such changes can also be used to update a local or remote data object with centralized availability values. With respect to each metabolite, updates in availability values may be summed to identify a total change and/or updated availability value. In some instances, limits are set with respect to a maximum change that may be effected across subsequent time steps and/or a maximum or minimum availability value for a metabolite.

At block 645, availability data is availed to a higher-level model. State vectors can then be updated based on data from multiple modules.

Some or all of blocks 620-645 may be repeated for each of multiple simulated time points in a simulation. Thus, at each time point, constraints can be updated based on state-vector information (e.g., representing availability of catalysts), an objective function can be defined (e.g., which may change across time points based on a configuration of a higher level objective), updated metabolite availability values can be determined, updated reaction fluxes can be identified, and further updated availability values can be determined. In some instances, a predefined number of simulated time points are to be evaluated and/or simulated time points corresponding to a predefined cumulative time-elapsing period are to be evaluated. In some instances, a subsequent simulated time point is to be evaluated until a predefined condition is satisfied. For example, a predefined condition may indicate that metabolite values for a current simulated time point are the same or substantially similar as compared to a preceding simulated time point or a preceding simulated time period.

With regard to a repeated iteration of block 630, it will be appreciated that an availability value determined for a given metabolite need not be equal to the corresponding updated availability value from the previous iteration of block 640 and/or the sum of the previously determined availability value adjusted by the identified flux pertaining to the metabolite. Rather, a processing of the previous time point with respect one or more other modules may have also resulted in a change in the metabolite availability, and/or a higher level constraint and/or processing may influence the availability. Thus, the availability value for a given metabolite determined at block 630 for a current time point may be equal to the availability value determined at block 630 for a preceding time point plus the cumulative updates to the availability value across modules, with any limits imposed.

While not shown in process 600, one or more variables can be output (e.g., transmitted to a user device). The variable(s) may include final values (e.g., availability values after all iterations have been performed), time-course values, high-level values and/or module-specific values. For example, the availability data may include, for each of one, more or all metabolites: an availability value (e.g., a final availability value) and/or a time course of the availability value. In some instances, the availability data is output with reference availability data. For example, when part or all of the processing performed to calculate the availability values was associated with a perturbation, the reference availability data may be associated with an unperturbed state. In some instances, a processed version of the availability data is output. For example, a comparison of availability values for particular metabolites across time points may be used to generate one or more growth metrics (e.g., a growth magnitude or rate), which may be output. Outputting the availability data can include (for example) locally presenting the availability data and/or transmitting the availability data to another device.

### III. Decomposing Biochemical Reactions and Processes

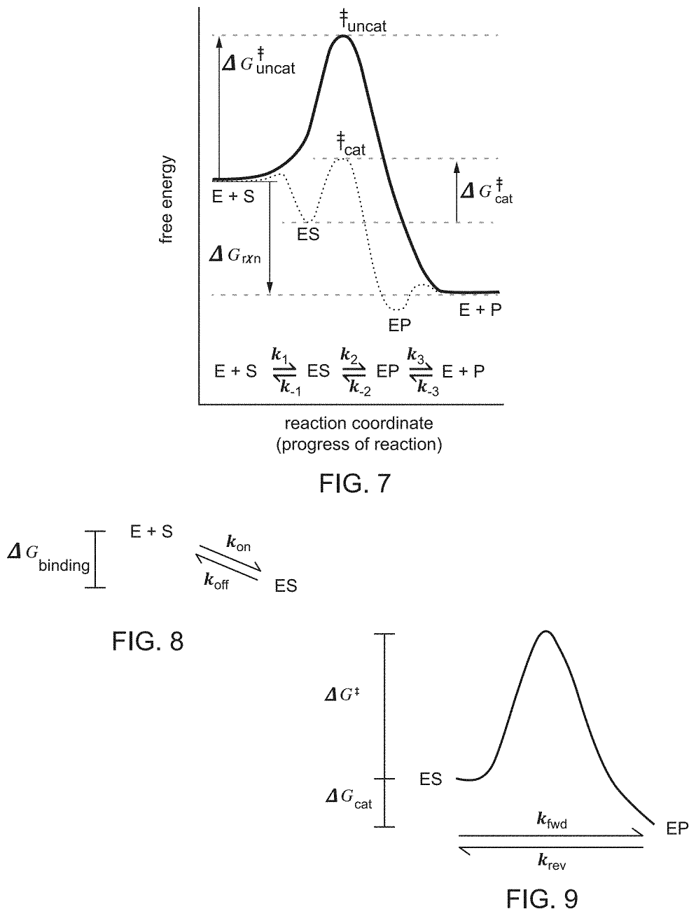

As shown in FIG. 7, the ability of enzymes (E) to catalyze reactions depends on their ability to interact directly and specifically with reactants. The reactant of an enzyme-catalyzed reaction is termed the substrate (S). An enzyme (E) works by binding to the substrate (S) to form an ES complex, then undergoing an internal state transition to an EP complex, which dissociates to release product (P) and regenerate free enzyme (E). The activation energy is lower for the ES ⇔EP transition than for uncatalyzed S ⇔P, which increases forward and reverse rates proportionally but does not affect overall ΔG. In other words, the enzyme (E) provides the chemical context for the bound substrate (S) to have access to a low energy path to formation of products (P). The transition from substrate (S) to product (P) results in a net change in energy content ΔG. The reaction passes through a transition state with activation energy ΔG ‡. The ΔG ‡ determines the rate of the reaction; where the higher the activation barrier, the slower the reaction. ΔG determines the ratio of the forward to reverse reaction rates, regardless of the activation barrier.

The mechanism of most enzymatic reactions can be broken down into three distinct steps including: (i) a binding-dissociation of the ES complex, (ii) a transition from the ES complex to the EP complex, and (iii) a binding-dissociation of the EP complex. Each of these steps can be considered as completely independent reversible reactions, each with its own free energy change and associated kinetics.

The binding-dissociation reactions may be described by a dissociation constant, K_{d}. The intuitive interpretation of K_{d} is the concentration of substrate (S) where the enzyme (E) is half bound. Whereas the physical meaning of K_{d} is related to the free energy of binding as expressed in Equation (2),

ΔG_{binding}=RT·ln K_{d} Equation (2)

where R is the ideal gas constant, T is absolute temperature, and K_{d} is the dissociation constant. The sign is chosen so that a smaller K_{d} corresponds to a more negative ΔG. For example, at K_{d}=1 mM, ΔG binding ≈−17 kJ. Binding-dissociation is an equilibrium between: (i) association, governed by a rate constant kₒₙ, and (ii) dissociation, governed by a rate constant k_{off} (also called k₁ and k₋₁), where K_{d} is equal to K_{off}/kₒₙ, as shown in FIG. 8 for the binding-dissociation of the ES complex. These binding-dissociation relationships between K_{d}, free energy ΔG, rate constant kₒₙ, and rate constant k_{off} are exploited herein to freely interconvert between K_{d}, free energy ΔG, on-rates, and off-rates.

Transition ES ⇔EP may be considered as an independent reaction, passing through a transition state. As shown in FIG. 9, there are two distinct energies to consider during transition: (i) the overall ΔG from ES to EP, and (ii) the activation energy ΔG ‡ that is a barrier between these states. The relationship between the activation energy ΔG ‡ and km comes from the Arrhenius equation—Equation (3),

K=Ae^{-ΔG‡/RT} Equation (3)

where k is the rate constant, A is a pre-exponential factor, ΔG ‡ is activation energy, R is the ideal gas constant, and T is absolute temperature. The overall ΔG of the reaction can be converted to the rate constant k by the same equation as for K_{d} above, which is equal to the ratio k_{fwd}/kᵣₑᵥ. Thus, as with the binding-dissociation reactions above, transition relationships between K_{d}, free energy ΔG, rate constant k_{fwd}, and rate constant kᵣₑᵥ are exploited herein to freely interconvert between free energy ΔG and forward and reverse rates.

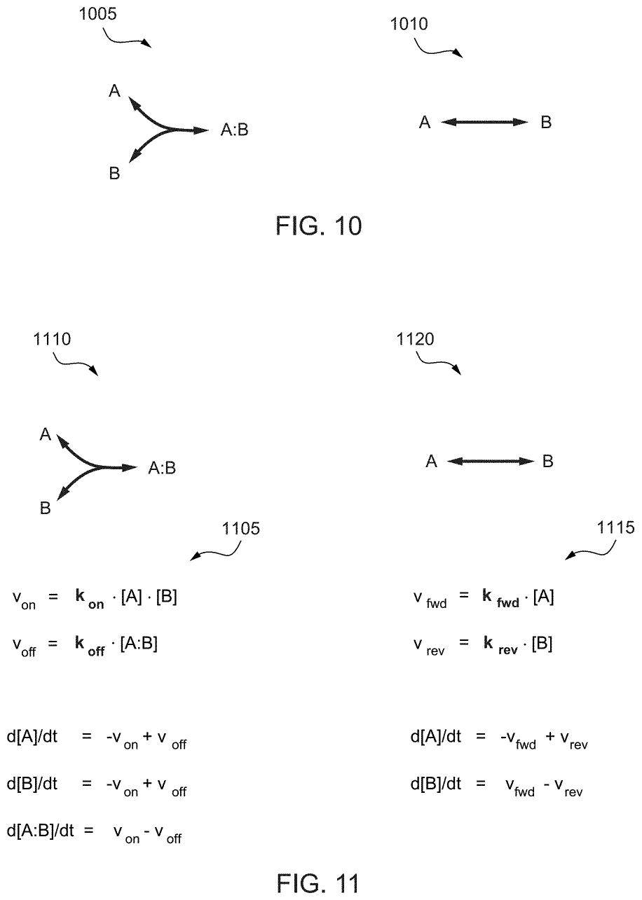

As shown in FIG. 10, the two basic components, a binding component 1005 and a transition component 1010, can be composed multiple ways to describe various enzymatic mechanisms of one or more reactions. The binding component 1005 comprises two molecules that associate or dissociate with some affinity. The transition component 1010 comprises the chemical conversion (forward and reverse) of one molecule to another through some transition state. Combinations of binding and transition components are very versatile in representing various enzymatic mechanisms of one or more reactions (a few common patterns cover most of the enzymatic mechanisms).

### IV. Modeling and Simulation of Biochemical Reactions and Processes

For modeling and simulation purposes described herein, there is a desire to predict a rate at which each reaction in a system is running in parallel given any condition. If it was possible to achieve a complete accounting of each of these rates, then it would be possible, in principle, to provide a complete detailed prediction of how the system will evolve over time (e.g., how concentrations of enzyme or product will change over time). Conventionally, the modeling and simulation of systems includes defining the detailed mechanisms for the system and attempting to find an analytical solution to produce one equation governing the overall behavior of the actions and/or components within the detailed mechanisms. However, an objective of the present disclosure is to model significantly larger and more complex systems (e.g., a whole cell), where such analytical solutions may not be possible, and where the approximations and assumptions conventionally used to find more local analytical solutions may not hold. Even well-established approximations such as Michaelis-Menten may not be appropriate for enzymes that are part of large, interconnected pathways.

Instead, various embodiments of the present disclosure are directed to techniques for modeling and simulating these systems, by: (i) systematically translating each component of a reaction into a set of rate equations to obtain a standard mathematical construct representing the individual binding or transition components regardless of the size of the biological system, and (ii) numerically integrating across the standard mathematical constructs using a system of ordinary differential equations to determine the contribution of each component to the rate of change of molecules with the mechanism or system. These techniques essentially provide a series of standardized mathematical constructs for each component that model the behavior of each component individually rather than providing a single algorithm that models the behavior of multiple components. Individually the standardized mathematical constructs are relatively easy to compute; and one of the few limitations to this approach is the amount of processing power available to compute multiple (tens, hundreds or thousands) standardized mathematical constructs in parallel to model or simulate the overall behavior of the mechanisms and system.

FIG. 11 shows a first standardized mathematical construct 1105 that may be used to model a single binding component 1110 (binding or dissociation) and a second standardized mathematical construct 1115 that may be used to model a single transition component 1120 (forward or reverse). Each rate equation of the standardized mathematical constructs has a simple form of one constant (kₒₙ, k_{off}, k_{fwd}, or kᵣₑᵥ) multiplied by a single concentration (e.g., [A], [B], or [A:B]) (a first order rate equation) or one constant (kₒₙ, k_{off}, k_{fwd}, or kᵣₑᵥ) multiplied by two concentrations (e.g., [A] and [B]) (second order rate equation). More specifically, the binding step of the binding component 1110 describes the association of one molecule [A] to one other molecule [B] to form a complex [A:B]. The forward rate vₒₙ (left to right, binding) is a function of the concentrations of both molecules, i.e. a second-order term. The dissociation step of the binding component 1110 describes the dissociation of the complex [A:B] into molecule [A] and the other molecule [B]. The reverse rate v_{off} (right to left, dissociation) is a function of the concentration of the complex [A:B], i.e., a first-order term. The transition step describes the chemical interconversion between two molecules [A] and [B]. The forward rate v_{fwd} and the reverse rate vᵣₑᵥ are both first-order terms, with the forward rate V_{fwd} depending on the left-hand molecule [A], and the reverse rate vᵣₑᵥ depending on the right-hand molecule [B]. Therefore, each component 1110; 1120 defines a single molecular event, contributing to a small number of terms in the ordinary differential equations describing the overall system. The power of this representation is that each type of component (binding or transition) is completely stereotypical, with kinetics described only in first and second order terms with respect to the system state. Nonetheless, the components 1110; 1120 can be composed into arbitrarily complex reaction mechanisms, following conventions commonly used in biochemistry to holistically model the overall behavior of systems. Advantageously, the total number of terms grows linearly as more components are added, and thus this technique scales easily in terms of size and complexity. Also advantageously, the standardized mathematical constructs are much simpler than conventional methods, which allows for reduced processor and memory constraints without a reduction in accuracy of modeling components.

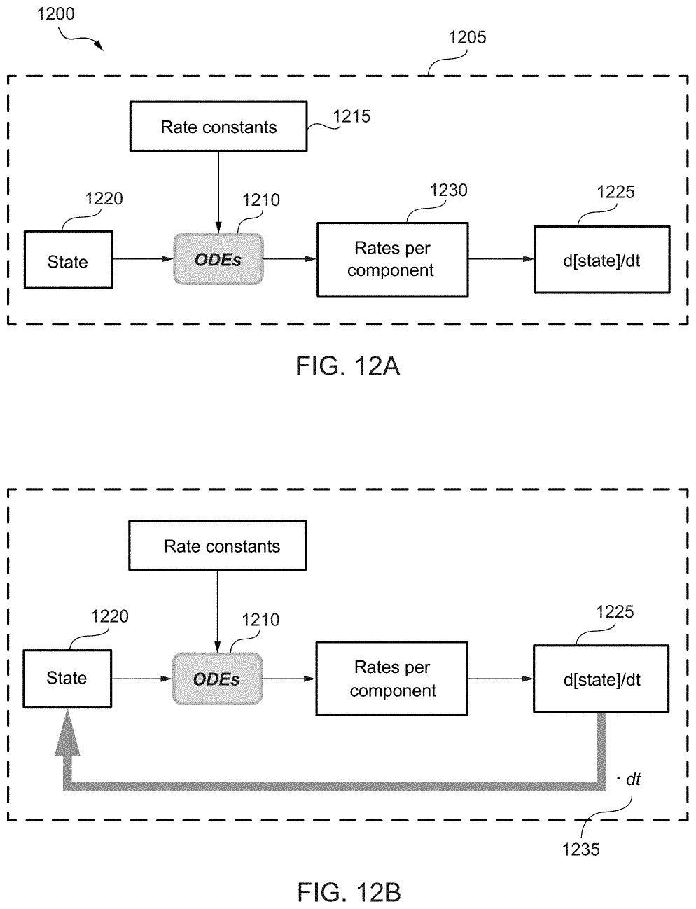

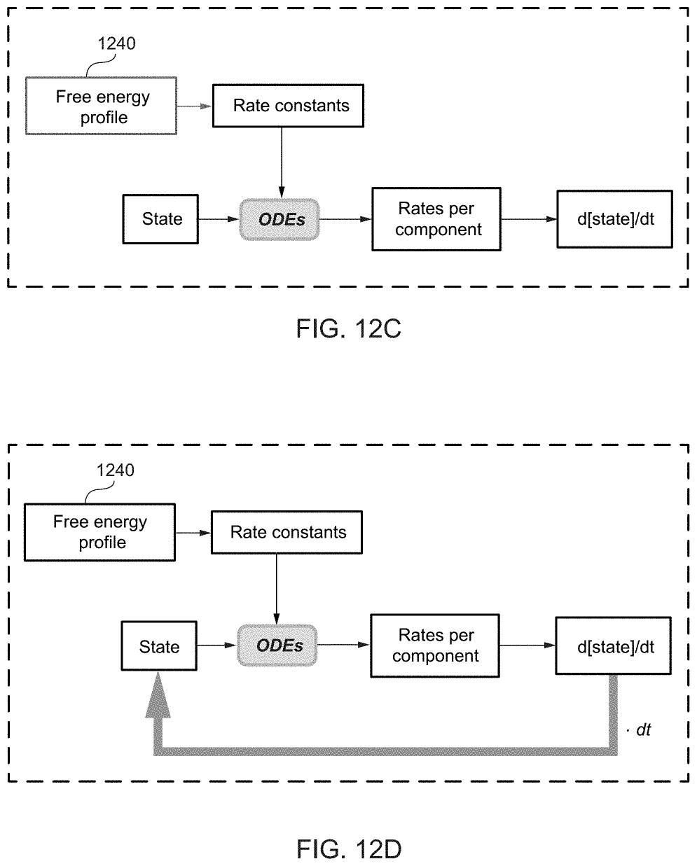

FIG. 12A shows a process flow for configuring components and sets of standardized mathematical constructs using an interaction system 1205 (e.g., interaction system 100 described with respect to FIG. 1) to simulate the behavior of a system (e.g., a system model such as the biological system model 200 described with respect to FIG. 2) by a simulation controller (e.g., simulation controller 300 described with respect to FIG. 3). Initially, a collection of related reactions, generally with shared molecules (e.g., enzymes, substrates or products), is defined for a kinetic pathway or mechanism(s) to be modeled, as described with respect to FIGS. 7-10. In some instances, higher-order attributes of the kinetic pathway or mechanism(s) may also be defined, such as which molecules are intermediates and which molecules are on the boundaries of the system. The configuration of the reactions and higher order attributes may comprise defining operations that translate relationships within the pathway or mechanism(s) into a system of ordinary differential equations 1210 (ODEs). The interaction system 1205 manages all of the operations to translate the kinetic pathway or mechanism(s) in to the system of ordinary differential equations 1210.

For example, each reaction of the kinetic pathway or mechanism(s) may be defined as an instance of a reaction class or object. Each instance of the reaction class or object may be defined via a list of instances of components, i.e., the component steps (binding, dissociation, and transition) of the overall reaction, and associated sets of standardized mathematical constructs that govern behavior of the components. The sets of standardized mathematical constructs defining the forward and reverse rates of binding, dissociation, and transition steps for the components in terms of first-order and second-order terms using concentrations of molecules in the system and forward and reverse rate constants 1215. The reaction class or object tracks a list of substrates, a list of products, and the enzyme that catalyzes the reaction. In some instances, the reaction class or object defines all of the intermediate forms of the enzyme, generally comprising the base enzyme bound to one or more substrates or products, as appropriate to the specific mechanism being modeled. Each component (binding or transition) within the list of instances of components defines a single molecular event, contributing to a small number of terms in a system of ordinary differential equations 1210 describing the overall system. A utility class or object may be used to map the variables of the kinetic pathway or mechanism(s) (e.g., concentrations of molecules such as concentration of enzyme, concentration of product, concentration of substrate, concentration of intermediates, etc.) calculated within the system of ordinary differential equations 1210 to and from numerical indices in an array of any number of dimensions such as a current state vector 1220 and a net rate of change vector.

The current state vector 1220 and the forward and reverse rate constants 1215 are input into the system of ordinary differential equations 1210. The system of ordinary differential equations 1210 may be represented by a single matrix built piecewise indicating the contribution of each component to the rate of change of each molecule 1225 of the system. The ordinary differential equations 1210 utilize the sets of standardized mathematical constructs to calculate the forward and reverse rates for each component within the instances of the reaction class or object as a function of the current state. The forward and reverse rates of each component are calculated independently, and then the forward and reverse rates for each component are summed to obtain the net rate of each step of the reaction 1230 (e.g., binding, dissociation, or transition). Each calculation follows mass-action kinetics, with terms that are either first-order or second-order, depending on the type of component. The ordinary differential equations 1210 numerically integrate across the set of standard mathematical constructs to determine the contribution of each component to the net rate of change of the molecules 1225 within the system based on the net rate of each step of the reaction 1230. The net rate of change of the molecules 1225 may be stored and maintained on a net rate of change vector.

FIG. 12B shows that if the model for the system is to be simulated over time, then the net rate of change 1225 for each molecule can be applied iteratively over some unit of time 1235 (i.e., a variable time step determined by the system based on concentrations of molecules in the system and a total time that the system is to evolve over (second, minutes, hours, etc.)) back to the current state vector 1220, which updates the current state vector 1220 to reflect the change of state of each molecule over the unit of time 1235. Consequently, the system of ordinary differential equations 1210 perform a numerical integration over the sets of standardized mathematical constructs for each unit of time 1235 that pulls together all the calculations into an instantaneous rate of change of all molecules in the system, which can be used to update a continuous number representing the concentration of each molecule on the current state vector 1220.

The standardized mathematical constructs (forward and reverse rates—vₒₙ, v_{off}, v_{fwd}, vᵣₑᵥ) of each component are governed by the rate constants 1215 (kₒₙ, k_{off}, k_{fwd}, or kᵣₑᵥ). More specifically, there is a forward rate constant (e.g., kₒₙ or k_{fwd}) and a reverse rate constant (e.g., k_{off} or kᵣₑᵥ) of each component, and these forwards and reverse rate constants 1215 are the parameters which must be supplied to build the model of the kinetic pathway or mechanism(s) for a system. The parametrization of the model with these forward and reverse rate constants 1215 can be error prone if the rate constants are treated individually because there is missing information or knowledge about how thermodynamics works in each of these mechanisms. Moreover, the rate constants 1215 (kₒₙ, k_{off}, k_{fwd}, or kᵣₑᵥ) cannot be looked up in published material and cannot be determined experimentally. Thus, techniques are required for representing these forward and reverse rate constants 1215 so that the rate constants 1215 can be used in the system of ordinary differential equations 1210 to simulate the system.

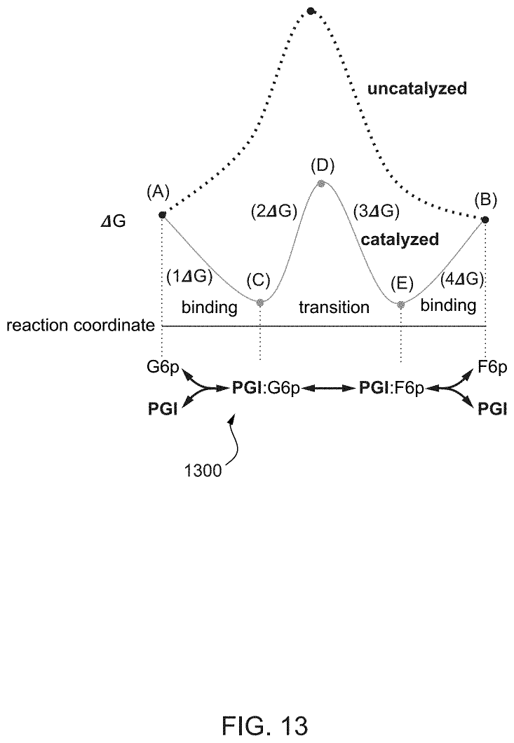

In various instances, techniques are disclosed for predicting these forward and reverse rate constants using free energy transitions (ΔG) for a given binding component or transition component. As shown in FIG. 13, the free energy states for a typical enzyme reaction 1300 comprise a first free energy state (A) for E+S and a second free energy state (B) for E+P, which are defined and unchangeable. Additionally, there are three free energy states between E+S and E+P (described herein as “waypoints” between E+S and E+P) comprising a third free energy state (C) for ES, a fourth free energy state (D) for the transition state between ES and EP, and a fifth free energy state (F) for EP, which are changeable to define a path from substrate to product (e.g., uncatalyzed path versus catalyzed path). Between these five free energy states there are free energy transitions (ΔG) that can be used to predict the forward and reverse rate constants (kₒₙ, k_{off}, k_{fwd}, or kᵣₑᵥ). The free energy transitions (ΔG) include: a first free energy transition (1ΔG) from E+S to ES, a second free energy transition (2ΔG) from ES to the transition state between ES and EP, third free energy transition (3ΔG) from the transition state between ES and EP to EP, and a fourth free energy transition (4ΔG) from EP to E+P.

The thermodynamics are given as input, represented as a free energy profile comprising a sequence of free energy transitions (ΔG) over the course of a component reaction. Each component may be treated differently for predicting the forward and reverse rate constants using free energy transitions (ΔG). In general the conversion of the sequence of free energy transitions (ΔG) to forward and reverse rate constants takes the form of the Arrhenius equation—Equation (4),

K=Ae^{+/-ΔG‡/RT} Equation (4)

where k is the rate constant, A is a pre-exponential factor, ΔG^{‡} is activation energy, R is the ideal gas constant, and T is absolute temperature. The exponential factors +/−1/RT and pre-exponential factor A may be built within the system of ordinary differential equations by the piecewise contribution of each component via calls to their respective exponential factors and pre-exponential factor methods with applicable reindexing.

Using the techniques described herein for predicting the forward and reverse rate constants using free energy transitions (ΔG), the process 1200 can be expanded to start with free energy profiles 1240 for each component from which it is possible to determine the forward and reverse rate constants, as shown in FIGS. 12C and 12D. However, there are a number of parameters for the free energy profiles 1240 (i.e., ΔG overall energy for each binding step; and ΔG^{‡} activation energy and ΔG overall energy for each transition step) that may not be known in some instances. Thus, additional techniques are disclosed herein for obtaining values for the free energy profiles 1240 in those instances to parameterize the model.

### V. Using Machine Learning to Fit Kinetic Parameters to Known Data

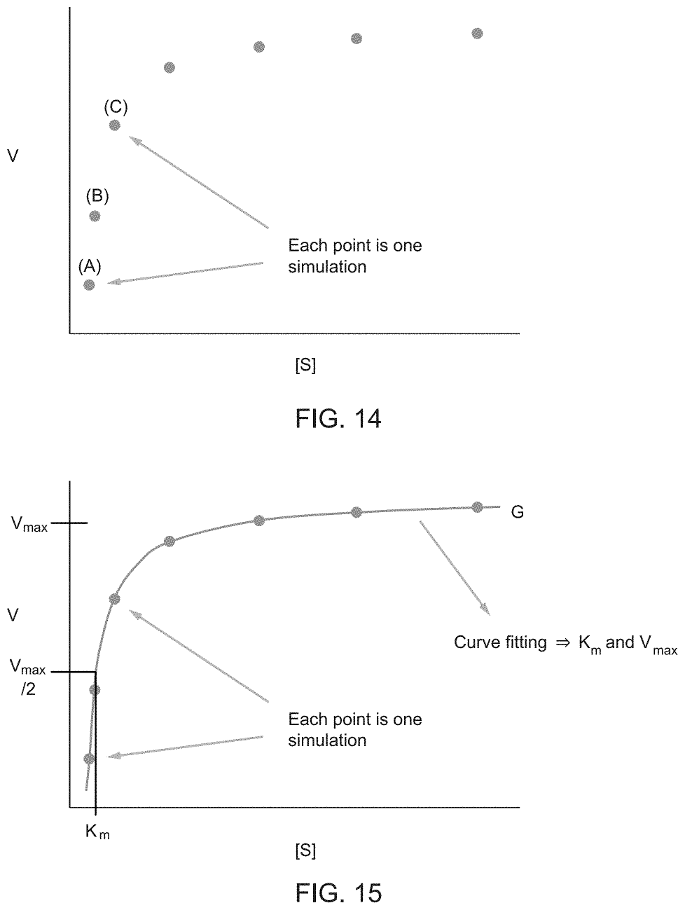

As shown in FIG. 14, if a concentration range for a substrate [S] (x-axis) is input into a model (as generated in accordance with FIGS. 1-10), then using the processes described with respect to FIGS. 12A-12D it is possible to determine the rate of a reaction V for each simulation when the model is run in silico. Each point on the graph represents a single simulation where the energy profile and forward and reverse rate constants are held invariant or constant and the concentration of substrate (current state vector) is varied over time (e.g., increased). For example, the model may be run in silico to obtain a first data point (A) representing a first simulation run with a first substrate concentration [S], a second data point (B) representing a second simulation run with a second substrate concentration [S+n], a third data point (C) representing a third simulation run with a third substrate concentration [S+n+m], and more of the same iteratively until a relationship is developed between the concentration of substrate [S] versus the rate of the reaction V.

As shown in FIG. 15, once enough data points are captured for the concentration range for the substrate [S], a best-fit line curve can be generated by the interaction system to connect the data points and graph the reaction rate V as a function of substrate concentration [S]. In some instances, the graph obtained will resemble the line G where the V will increase rapidly at low substrate concentrations [S], then level off to a flat plateau at high substrate concentrations [S]. The plateau occurs because the enzyme is saturated, meaning that all available enzyme molecules are already tied up processing substrates. Any additional substrate molecules will simply have to wait around until another enzyme becomes available, so the rate of reaction (amount of product produced per unit time) is limited by the concentration of enzyme. This maximum rate of reaction is characteristic of a particular enzyme at a particular concentration and is known as the maximum velocity Vₘₐₓ and is typically known for most enzymes (i.e., the Y-value (initial rate of reaction value) at which the graph above plateaus). The substrate concentration that gives a rate that is halfway to Vₘₐₓ is called the Kₘ (Michaelis constant), and is a useful measure of how quickly reaction rate increases with substrate concentration. Kₘ is also a measure of an enzyme's affinity for (tendency to bind to) its substrate. A lower Kₘ corresponds to a higher affinity for the substrate, while a higher Kₘ corresponds to a lower affinity for the substrate. Unlike Vₘₐₓ, which depends on enzyme concentration, Kₘ is always the same for a particular enzyme characterizing a given reaction and is typically known for most enzymes.

The ability to develop a relationship between the concentration of substrate [S] versus the rate of the reaction V based on free energy profiles to obtain values for Vₘₐₓ and Kₘ forms the basis of a design for a machine learning approach in accordance with aspects of the present disclosure. In various embodiments, the machine learning approach comprises: (i) defining a system to be simulated in terms of binding and transition steps, (ii) learning ΔG overall energy for each binding step and ΔG^{‡} activation energy and ΔG overall energy for each transition step subject to constraints on their sum across each overall reaction, (iii) translating, at each evaluation of the system, the various parameters to kₒₙ, k_{off}, k_{fwd}, or i rate constants, (iv) determining steady state concentrations of all molecules including intermediates and corresponding fluxes by solving a resulting system of ordinary differential equations, and (v) evaluating loss against known Vₘₐₓ and Kₘ parameters.

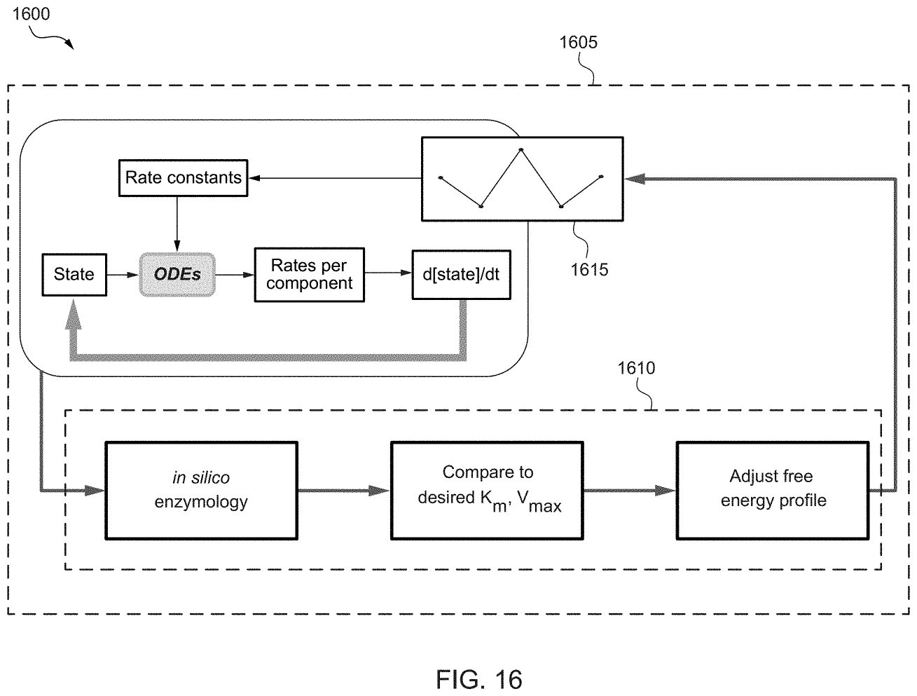

FIG. 16 shows a process flow 1600 for configuring components and sets of standardized mathematical constructs using an interaction system 1605 (e.g., interaction system 100 described with respect to FIG. 1) to simulate the overall behavior of a system (e.g., a system model such as the biological system model 200 described with respect to FIG. 2) by a simulation controller (e.g., simulation controller 300 described with respect to FIG. 3) implementing a prediction model 1610. Initially, a collection of related reactions, generally with shared molecules (e.g., substrates or products), is defined for a kinetic pathway or mechanism(s) to be modeled, and an energy profile 1615 is obtained for each reaction. The defining the collection of reactions for the system to be modeled may comprise defining the expected behavior for each reaction. The expected behavior may be defined based on the molecules used within each component step. For example, Vₘₐₓ and Kₘ, which define the expected behavior for given molecules, can be obtained from a knowledge bank or data storage and used to define the behavior of each component step that includes the given molecules. The energy profiles 1615 comprise the free energy state ΔG for each component step, e.g., ΔG overall energy for each binding step and ΔG^{‡} activation energy and ΔG overall energy for each transition step. The energy profiles 1615 may be initially obtained arbitrarily (e.g., selected by a user or the integration system randomly or without reason), rationally (e.g., selected by a user or the integration system with some reasoning), explicitly (e.g., selected by a user or the integration system from a library of energy profiles developed for the same binding or transition step), or a combination thereof.

Using the processes described with respect to FIGS. 12A-12D it is possible to calculate the forward and reverse rate constants (kₒₙ, k_{off}, k_{fwd}, or kᵣₑᵥ) based on the obtained energy profiles, calculate the forward and reverse rates of each component independently using the forward and reverse rate constants (kₒₙ, k_{off}, k_{fwd}, or kᵣₑᵥ) and the standardized mathematical constructs (vₒₙ, v_{off}, v_{fwd}, vᵣₑᵥ), and then the forward and reverse rates for each component are summed to obtain the net rate of each step of the reaction (e.g., binding, dissociation, or transition). The system of ordinary differential equations numerically integrates across the set of standard mathematical constructs to determine the contribution of each component to the net rate of change of each molecule within the system based on the net rate of each step of the reaction. The in silico behavior of the system derived from the current state vector, the net rate of each step of the reaction, and the net rate of change of each molecule is provided to the prediction model 1610 as input data for determining the Vₘₐₓ and Kₘ of the current simulation for each reaction, comparing the Vₘₐₓ and Kₘ for the in silico behavior of each reaction to the known Vₘₐₓ and Kₘ for the expected behavior of each reaction, and predicting a new energy profile based on a fitting of the in silico behavior to the expected behavior, and adjusting energy profiles 1615 accordingly.

The prediction model 1610 can be a machine-learning model, such as a neural network, a convolutional neural network (“CNN”), e.g. an inception neural network, a residual neural network (“Resnet”) or NASNET provided by GOOGLE LLC from MOUNTAIN VIEW,

CALIFORNIA, or a recurrent neural network, e.g., long short-term memory (“LSTM”) models or gated recurrent units (“GRUs”) models. The prediction model 1610 can also be any other suitable machine-learning model trained to predict energy profiles for a reaction, such as a support vector machine, decision tree, a regression model, coarse-grained models, normal mode analysis, or Markov state models, random forest classifier, a three-dimensional CNN (“3DCNN”), a dynamic time warping (“DTW”) technique, a hidden Markov model (“HMM”), etc., or combinations of one or more of such techniques—e.g., CNN-HMM or MCNN (Multi-Scale Convolutional Neural Network).

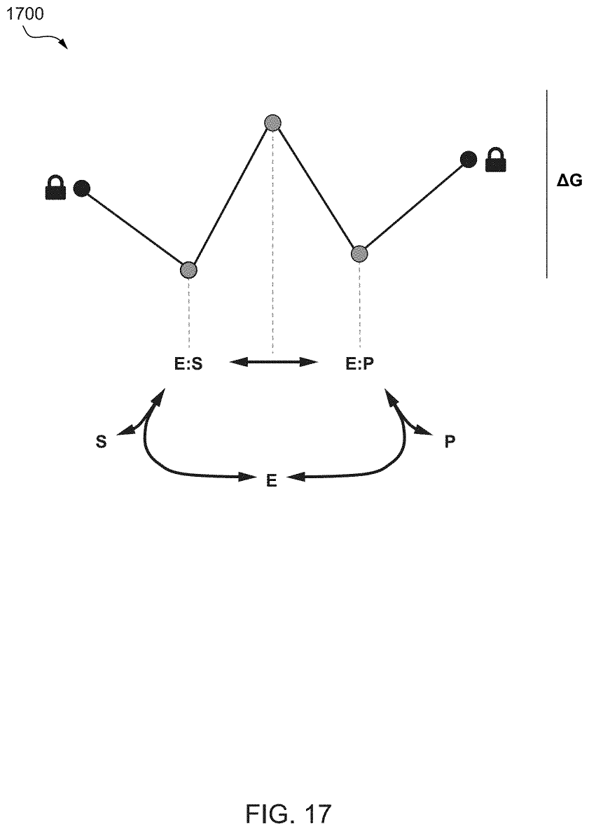

To train the prediction model 1610, training samples are obtained or generated. The training samples can include a current state vector, a net rate of each step of the reaction, and a net rate of change of each molecule for various component steps, and optional labels corresponding to energy profile parameters such as ΔG overall energy for each binding step and ΔG^{‡} activation energy and ΔG overall energy for each transition step (including: a first free energy transition (1ΔG) from E+S to ES, a second free energy transition (2ΔG) from ES to the transition state between ES and EP, third free energy transition (3ΔG) from the transition state between ES and EP to EP, and/or a fourth free energy transition (4ΔG) from EP to E+P). In some instances, the training process includes iterative operations to find a set of parameters for the prediction model 1610 that minimizes a loss function for the prediction models 1610. Each iteration can involve finding a set of parameters for the prediction model 1610 so that the value of the loss function using the set of parameters is smaller than the value of the loss function using another set of parameters in a previous iteration. The set of parameters may comprise the waypoints or free energy states between E+S and E+P comprising a free energy state (A) for ES, a free energy state (B) for the transition state between ES and EP, and a free energy state (C) for EP, which are changeable to define a path from substrate to product (e.g., uncatalyzed path versus catalyzed path), as shown in FIG. 17. The loss function can be constructed to measure the difference between the outputs predicted using the prediction models 1610 (e.g., ΔG overall energy for each binding step and ΔG^{‡} activation energy and ΔG overall energy for each transition step) and the optional labels contained in the training samples. In some instances, the loss function may be constructed to measure this difference based on the comparison between the determined Vₘₐₓ and Kₘ of the current simulation using a given or predicted energy profile and the known Vₘₐₓ and Kₘ for the expected behavior of a reaction. Once the set of parameters are identified, the prediction model 1610 has been trained and can be tested, validated, and/or utilized for prediction in simulations as designed.

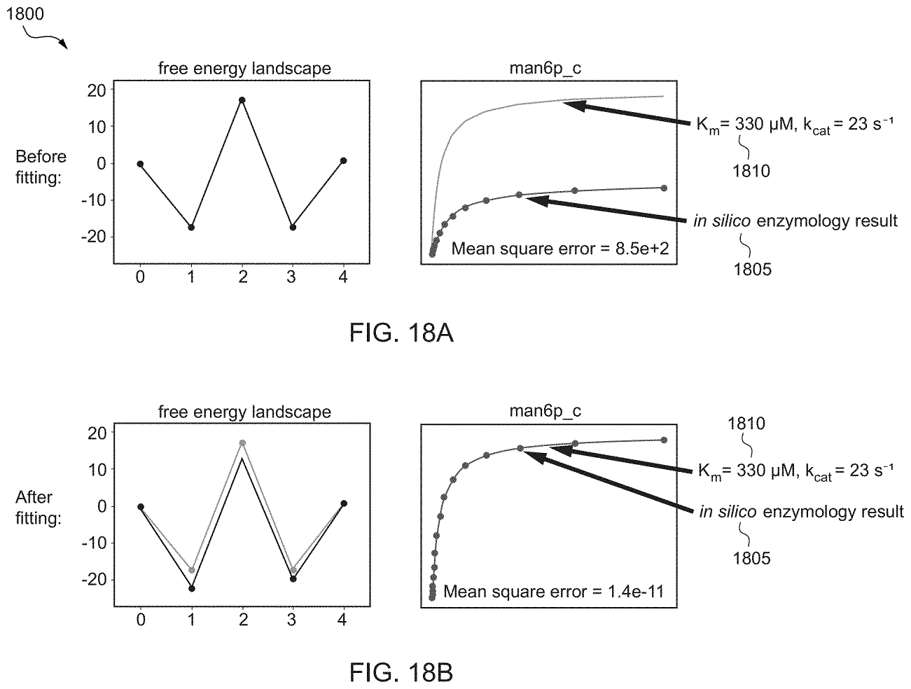

In some instances, prior to a fitting and prediction by the prediction model 1610, the simulation of a model for a system may be run iteratively to obtain in silico behavior of each reaction of the collection of reactions based on an initial energy profile and derive the reaction rate of each reaction as a function of substrate concentration. As shown in FIG. 18A, the in silico behavior 1805 of a reaction determined based on the initial energy profile obtained prior to a fitting and prediction by the prediction model 1610 may be far from meeting the expected behavior 1810 of the reaction based on a comparison between the Vₘₐₓ and Kₘ for the in silico behavior of the reaction and the known Vₘₐₓ and Kₘ for the expected behavior of the reaction. Consequently, the in silico behavior 1805 including the current state vector, the net rate of each step of the reaction, and the net rate of change of each molecule may be provided to the prediction model 1610 as input data for predicting a new energy profile for the reaction based on the fitting of the in silico behavior to the expected behavior and the loss function. The predicting may comprise learning a set of parameters including the waypoints or free energy states between E+S and E+P, which are changeable to define a path from substrate to product to ultimately fit the in silico behavior to the expected behavior. The predicting may further comprise outputting a new energy profile comprising a ΔG overall energy for each binding step and ΔG^{‡} activation energy and ΔG overall energy for each transition step (e.g., including: a first free energy transition (1ΔG) from E+S to ES, a second free energy transition (2ΔG) from ES to the transition state between ES and EP, third free energy transition (3ΔG) from the transition state between ES and EP to EP, and/or a fourth free energy transition (4ΔG) from EP to E+P).

After a fitting and prediction by the prediction model 1610, the simulation of the model for the system may again be run iteratively to obtain in silico behavior of each reaction of the collection of reactions based on the new energy profile and derive the reaction rate of each reaction as a function of substrate concentration. As shown in FIG. 18B, the in silico behavior 1805 of a reaction determined based on the new energy profile obtained after the fitting and prediction by the prediction model 1610 may be substantially closer to meeting the expected behavior 1810 of the reaction based on a comparison between the Vₘₐₓ and Kₘ for the in silico behavior of the reaction and the known Vₘₐₓ and Kₘ for the expected behavior of the reaction. In some instances, if the in silico behavior still does not meet the expected behavior of the reaction, then the in silico behavior 1805 including the current state vector, the net rate of each step of the reaction, and the net rate of change of each molecule may be provided to the prediction model 1610 as input data for predicting an updated energy profile for the reaction based on the fitting of the in silico behavior to the expected behavior and the loss function. In some instances, whether the in silico behavior meets the expected behavior of the reaction may be determined when the Vₘₐₓ and Kₘ for the in silico behavior of the reaction is equal to or substantially equal to the known Vₘₐₓ and Kₘ for the expected behavior. The training, predicting, and fitting processes may be performed iteratively until the in silico behavior meets the expected behavior.

### VI. Multi-Substrate Reactions

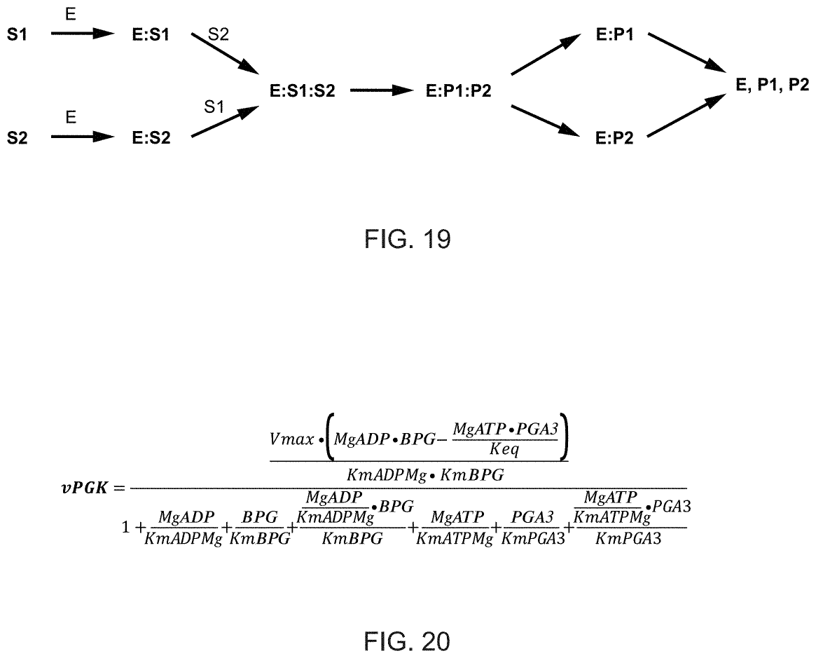

Enzymatic reactions may include an enzyme binding to more than one substrate. FIG. 19 illustrates an example of a multi-substrate enzymatic reaction. FIG. 19 shows two substrates, S1 and S2. In this example, S1 and S2 are different substrates, but in some embodiments, S1 and S2 may be the same substrate. The enzyme E may bind to either one of the substrates. The resulting complex, either E:S1 or E: S2, binds to the remaining substrate to form a multi-substrate complex, E:S1:S2. The multi-substrate complex then transitions to E:P1:P2. In this example, P1 and P2 are different products, but in some embodiments, P1 and P2 may be the same product. The multi-substrate complex releases either P1 or P2, leaving a complex of E:P2 or E:P1, respectively. The two complexes, E:P1 or E:P2, then disassociate to release enzyme E, resulting in products P1 and P2, along with unbound E.

The mathematics of a multi-substrate enzymatic reaction are more complicated than a single-substrate enzymatic reaction. FIG. 20 shows an example of a Michaelis-Menten rate equation for a multi-substrate reaction with the phosphoglycerate kinase step in glycolysis. The reaction involves two substrates, two enzymes, and two products. As can be seen from this equation, more terms are in the rate equation than for a single-substrate reaction (e.g, FIG. 7). The microscopic rate constants are not shown in the figure, but the figure shows the effect of more molecules with the macroscopic constants, Vmax and a Km for each substrate and product. The microscopic rate constants for multi-substrate reactions are even harder to determine experimentally than for single-substrate reactions at least because two microsteps (i.e., E binding to S1, E:S1 binding to S2) may need to be observed instead of just one (i.e., E binding to S1). Frequently, each of the rate constants are not determined thereby causing more unknowns to be involved. Simplifying assumptions used for single substrate reactions, which include assuming some rate constants are much smaller than other rate constants, may or may not be applicable. Conventionally, assuming a rate constant to be much smaller than other rate constants essentially zeroes out a rate constant and allows for an entire microstep to be removed from consideration. A conventional simplifying assumption is that the rate of an enzyme-substrate dissociating is much larger than forming the enzyme-product complex. With multi-substrate enzymatic reactions, the appropriate simplifying assumptions are not clear, as there are multiple enzyme-substrate complexes and multiple enzyme-product complexes. Additionally, given that two substrates may result in eight additional rate constants solely on the substrate side, a simplifying assumption that eliminates one or two microsteps may not significantly simplify the rate equations for conventional modeling.

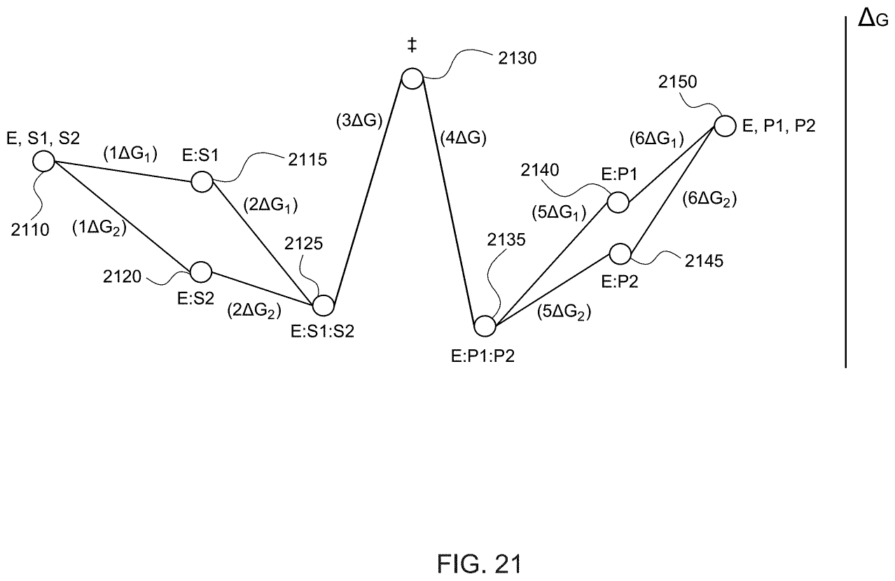

FIG. 21 shows a free energy landscape for a multi-substrate enzymatic reaction. The free energy landscape, also called a free energy profile, is illustrated as paths between different “waypoints” (shown as circles). Waypoints illustrate the free energy state after a certain point (e.g., at the end of an microstep) in the overall reaction. Waypoint 2110 represents the free energy for unbound enzyme E, substrate S1, and substrate S2. Waypoint 2115 represents the free energy of a complex E:S1 of enzyme E bound to substrate S1, along with unbound substrate S2. Waypoint 2120 represents the free energy of a complex E:S2 of enzyme E bound to substrate S2, along with unbound substrate Si.

Waypoint 2125 represents the free energy of a multi-substrate complex E:S1:S2. The multi-substrate complex is formed by both substrates binding to the enzyme. Waypoint 2130 represents the free energy of a catalyzed transition complex, analogous to the catalyzed transition complex in FIG. 7. Waypoint 2135 represents the free energy of a multi-substrate complex E:P1:P2, where enzyme E is bound to product P1 and product P2.

Waypoint 2140 represents the free energy of complex E:P1 and unbound byproducts. Complex E:P1 may be formed by dissociation of P2 from E:P1:P2. Additionally, some byproducts (e.g., CO₂ or H₂O) may become unbound when the E:P1 complex forms. Waypoint 2145 represents the free energy of complex E:P2 and unbound byproducts. Complex E:P2 may be formed by dissociation of P1 from E:P1:P2. Additionally, some byproducts (e.g., CO₂ or H₂O) may become unbound when the E:P2 complex forms.

Waypoint 2150 represent the free energy of unbound E, P1, and P2. Unbound E, P1, and P2 are formed after the remaining product dissociates from the enzyme. Waypoint 2150 may also include free energy of unbound byproducts.

In some embodiments, the free energy of waypoint 2110 and waypoint 2150 are known. The difference between waypoint 2110 and waypoint 2150 is ΔG for the reaction and may be determined theoretically or by experiment. Initially, the free energies of the other waypoints are not known. However, the free energies of these inner waypoints may be estimated or approximated. The free energies may be estimated based on free energies associated with other substrates and enzymes. The general shape of the free energy landscape may be known to decrease to the E:S1:S2 (waypoint 2125) or other multi-substrate complex. The catalyzed transition complex at waypoint 2130 may be estimated to be relatively high compared to waypoint 2125 and 2135.

The initial free energy profile may be used to model the enzymatic reaction, similar to the process illustrated in FIG. 16. For a free energy at a waypoint, the equilibrium constant K_{d} for a reaction can be calculated and therefore also the ratio of the rate constants, as explained with Equation (3) above. Any combination of k_{off} and kₒₙ that is consistent with K_{d} (equal to k_{off}/kₒₙ) may be consistent with the free energy at the waypoint. A constant value can be assumed for kₒₙ or k_{off} and the other rate constant can be calculated from K_{d}. If one of the rate constants kₒₙ or k_{off} is known, then the other rate constant can be calculated from K_{d}. The rate constants can then be determined, as described with FIGS. 12A-12D. With the rate constants (which may be determined using either assumed or measured rate constants), the overall reaction can be simulated. The simulation of the overall reaction can generate velocities (e.g., V in FIG. 14) of the overall reaction for different substrate concentrations (e.g., [S] in FIG. 15).

For the generated rates of the overall reaction for different substrate concentration, the simulated catalytic rate constant k_{cat} and Michaelis constant Kₘ can be determined. The simulated k_{cat} and Kₘ can be compared to a known k_{cat} and Kₘ, which may be obtained experimentally. The free energy profile may be adjusted to reduce or minimize the difference between the simulated k_{cat} and Kₘ and the known k_{cat} and Kₘ. The adjusting of the free energy profile may be similar to prediction model 1610 in FIG. 16 but with two substrates and two products and the waypoints associated with a multi-substrate reaction.

To train the prediction model 1610 for a multi-substrate enzymatic reaction, training samples may be obtained or generated. The training samples can include a current state vector, a net rate of each step of the reaction, and a net rate of change of each molecule for various component steps, and optional labels corresponding to energy profile parameters such as ΔG overall energy for each binding step and ΔG activation energy and ΔG overall energy for each transition step (including: a first free energy transition (1ΔG₁) from E+S₁ to E:S1, another first free energy transition (1ΔG₂) from E+S2 to E:S₂, a second free energy transition (2ΔG₁) from E:S1+S2 to E:S1:S₂, another second free energy transition (2ΔG₂) from E:S2+S1 to E:S1:S2, a third free energy transition (3ΔG) from E:S1:S2 to the transition state between E:S1:S2 and E:P1:P2, a fourth free energy transition (3ΔG) from the transition state between E:S1:S2 and E:P1:P2 to E:P1:P2 , a fifth free energy transition (5ΔG₁) from E:P1:P2 to E:P1+P2, another fifth free energy transition (5ΔG₂) from E:P1:P2 to E:P2+P1, a sixth free energy transition (6ΔG₁) from E:P1+P2 to E+P1+P2, another sixth free energy transition (6ΔG₂) from E:P2+P1 to E+P 1+P2).

In some instances, the training process includes iterative operations to find a set of parameters for the prediction model 1610 that minimizes a loss function for the prediction models 1610. Each iteration can involve finding a set of parameters for the prediction model 1610 so that the value of the loss function using the set of parameters is smaller than the value of the loss function using another set of parameters in a previous iteration. The set of parameters may include the waypoints or free energy states between E+S1+S2 and E+P1+P2 (e.g., all waypoints shown in FIG. 21 except for waypoints 2110 and 2150), which are changeable to define a path from substrate to product. The loss function can be constructed to measure the difference between the outputs predicted using the prediction models 1610 (e.g., ΔG overall energy for each binding step and ΔG^{‡} activation energy and ΔG overall energy for each transition step) and the optional labels contained in the training samples. In some instances, the loss function may be constructed to measure this difference based on the comparison between the determined Vₘₐₓ (or k_{cat}) and Kₘ of the current simulation using a given or predicted energy profile and the known Vₘₐₓ (or k_{cat}) and Kₘ for the expected behavior of a reaction. Once the set of parameters are identified, the prediction model 1610 has been trained and can be tested, validated, and/or utilized for prediction in simulations as designed.

In some instances, prior to a fitting and prediction by the prediction model 1610, the simulation of a model for a system may be run iteratively to obtain in silico behavior of each reaction of the collection of reactions based on an initial energy profile and derive the reaction rate of each reaction as a function of substrate concentration. The fitting and predicting may be similar to fitting and predicting for a single-substrate, where the simulated behavior is compared to an expected behavior iteratively, as described with FIGS. 18A and 18B.

FIG. 21 is only one example of a free energy profile. Free energy profiles may have a different number of waypoints, which may be more or less. An enzymatic reaction with more intermediate steps may have more waypoints. An enzymatic reactions with more enzymes and more substrates will likely have more intermediate steps. In some embodiments, a specific substrate may react with a specific enzyme. In some embodiments, a specific substrate may react with multiple enzymes, or a specific enzyme may react with multiple substrates. Free energy profiles may also have a different shape than shown in FIG. 21. For example, the waypoints are not restricted to being relatively higher or lower than other waypoints as shown in FIG. 21. In some embodiments, the transition waypoint (e.g., waypoint 2130) may be a local maximum. The enzyme:substrate complex waypoint (e.g., waypoint 2125) or the enzyme:product waypoint (e.g., waypoint 2135) may be local minima and have free energies less than their dissociated components; the complexes are expected to have lower free energy such that the complexes are favored thermodynamically to form rather than to repel each other.

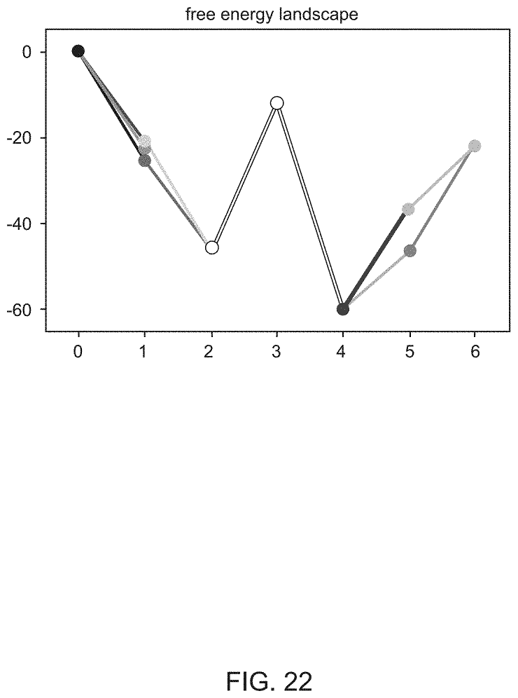

FIG. 22 is an example of a free energy landscape or a free energy profile of part of the threonine biosynthesis pathway. The reaction includes two substrates and two products (e.g., a bi-bi reaction). The change in free energy is shown on the y-axis, with zero designating the free energy of the initial unbound substrate and products. The x-axis shows numbering of the reaction coordinates from 0 to 6. Reaction coordinate 0 represents the initial unbound substrate and products. Reaction coordinate 1 represents the free energies two complexes: a first complex with the enzyme bound to a first substrate, and a second complex with the enzyme bound to a second substrate. Also at reaction coordinate 1 is an inhibitor (threonine). The inhibitor is modeled as unproductive binding: E+I↔E:I. The free energy profile allows for these three multiple parallel paths at reaction coordinate 1. Reaction coordinate 2 represents the enzyme bound with the first substrate and the second substrate. Reaction coordinate 3 represents the catalyzed transition state. Reaction coordinate 4 represents the enzyme bound with the first product and the second product. Reaction coordinate 5 represents the free energy of two different complexes with products: a first complex with the enzyme bound to the first product, and a second complex with the enzyme bound to the second product. Reaction coordinate 6 represents the unbound enzyme, first product, and second product. The free energy profile in FIG. 22 is a profile that minimizes the differences between the simulated k_{cat} and Kₘ and the known k_{cat} and Kₘ. The general shape of this free energy profile may be used as an initial free energy profile for other multi-substrate enzymatic reactions.

### VII. Example Methods

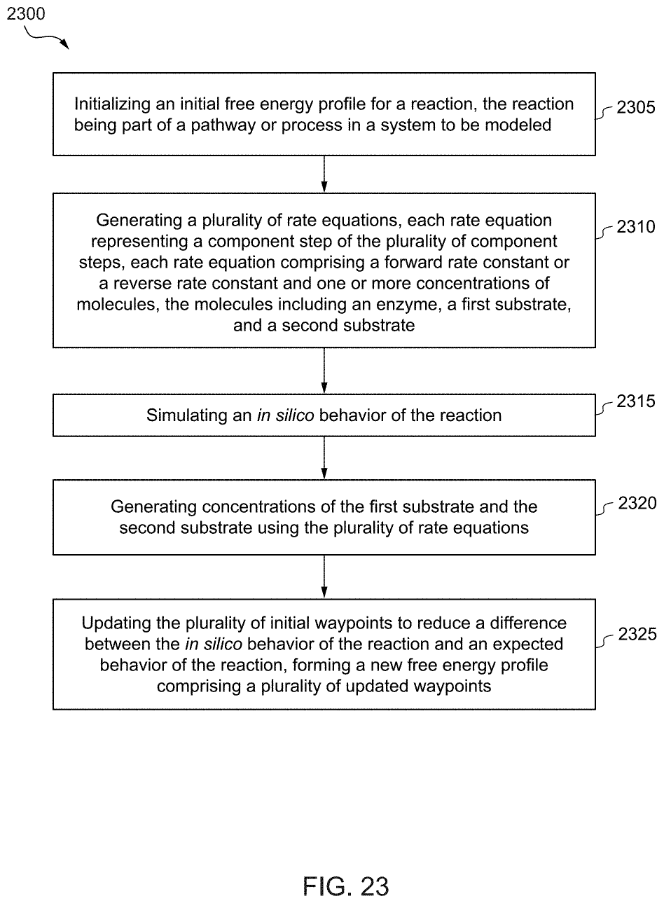

FIG. 23 illustrates a process 2300 for modeling enzymatic reactions, including any reaction disclosed herein. The reaction may be part of a pathway or process in a system to be modeled. The system may include a biological cell, such as E. coli. The pathway may include any metabolic pathway or cycle, such as the citric acid cycle. The system may represent a biological system, including core metabolism, membrane synthesis, cell-wall synthesis, DNA replication, transcription, transcription regulation, translation, RNA salvage, protein and RNA maturation, protein salvage, transmembrane transport (including electron chain, oxidative phosphorylation, redox, and pH interconversion activity), signal transduction, stress response and growth rate regulation (SOS), cell division, chemotaxis, and cell-cell signaling. The reaction may be related to any biological function shown in FIG. 2. The process may be performed by simulation controller 105.

At block 2305, process 2300 may include initializing an initial free energy profile for reaction. The initial free energy profile may include a plurality of initial waypoints. For example, the initial waypoints may be the waypoints in FIG. 21. Each initial waypoint may represent a free energy state. The reaction may involve an enzyme, a first substrate, and a second substrate. The reaction may involve an enzyme, a first product, and a second product. As a reaction may run in a reverse direction, a substrate may be a product, and a product may be a substrate. Reactions may include more than two substrates or products. Reactions may include two or more enzymes. The initial free energy profile may include parallel paths to reach some or all of the initial waypoints between the ends of the reaction. A waypoint may represent a microscopic step after a species has been replaced with another species (e.g., an enzyme:substrate complex is replaced with an enzyme:product complex).

The reaction may include a plurality of components steps. A component step may be a microstep in the overall enzymatic reaction. The sum or completion of the microsteps may be the overall reaction. For example, each line connecting waypoints in FIG. 21 may be considered a microstep or a component step. An overall enzymatic reaction may include (for example) at least 2, 3, 4, 5, 6, 10, 15, or 20 microsteps and an equivalent number of component steps. The method may apply to any model described herein and models described in U.S. application Ser. No. 16/942,222, filed Jul. 29, 2020, the entire contents of which are incorporated herein by reference for all purposes.

Each end of a component step of the plurality of components steps may correspond to an initial waypoint of the plurality of initial waypoints in the initial free energy profile. The free energy profile may be of Gibbs free energy. A first initial waypoint of the plurality of initial waypoints may represent a first free energy state of a multi-substrate complex of the enzyme bound to the first substrate and to the second substrate. The multi-substrate complex may be a tripartite complex. The multi-substrate complex may be E:S1:S2 as shown in FIG. 21.

The plurality of component steps may include a first binding component step and a second binding component step. The first binding component step may include binding between the enzyme and the first substrate to form an enzyme:first substrate complex. As an example, enzyme:first substrate may be E:S₁ in FIG. 21. The enzyme:first substrate complex may correspond to an initial waypoint. The second binding component step may include binding between the enzyme and the second substrate to form an enzyme:second substrate complex. An example of enzyme:second substrate complex is E:S2 in FIG. 21. The enzyme:second substrate complex may correspond to another initial waypoint.

The plurality of components steps may include a third binding component step and a fourth binding component step. The third binding component step may include binding between the enzyme:first substrate complex and the second substrate to form the multi-substrate complex. For example, the third binding component step may be represented as the line between waypoint 2115 to waypoint 2125. The fourth binding component step may include binding between the enzyme:second substrate and the first substrate. As an example, the fourth binding component may be represented as the line between waypoint 2120 and waypoint 2125. The end of the third binding component step and the end of the fourth binding component step may be the first initial waypoint (e.g., waypoint 2125).

The plurality of component steps may include a transition component step. The transition component step may include a chemical conversion of the multi-substrate complex to a multi-product complex (e.g., waypoint 2130 in FIG. 21). The multi-product complex may be E:P1:P2 in FIG. 21. The multi-product complex may correspond to another initial waypoint (e.g., waypoint 2135 in FIG. 21). The multi-product complex may include a first product and a second product. The first product and the second product may be the same or different.

The plurality of component steps may include a first dissociation component step and a second dissociation component step. The first dissociation component step may include a dissociation of the multi-product complex into a first complex of the enzyme and the first product (e.g., E:P1 in FIG. 21). The second dissociation component step may include a dissociation of the multi-product complex into a second complex of the enzyme and the second product (e.g., E:P2 in FIG. 21).

The plurality of component steps may include a third dissociation component step and a fourth dissociation component step. The third dissociation component step may include a dissociation of the first complex into unbound enzyme E and unbound product P1. The fourth dissociation component step may include a dissociation of the second complex into unbound enzyme E and unbound product P2. The end of both the third dissociation component step and the fourth dissociation component step may be a waypoint representing the free energy of unbound enzyme E, product P1, and product P2 (e.g., waypoint 2150 in FIG. 21).

Additional substrates and/or enzymes may increase the number of component steps, including binding component steps, transition component steps, and disassociation component steps. The number of waypoints may then also increase.

At block 2310, process 2300 may include generating a plurality of rate equations. The rate equation may describe the rate of change of a molecule's concentration. Each rate equation may represent a component step of the plurality of components steps. Each rate equation may include a forward rate constant or reverse rate constant and one or more concentrations of molecules. Rate equations may be any rate equation disclosed herein. The molecules may include the enzyme, the first substrate, and the second substrate.

The rate equations may have a form of a forward rate constant k_{fwd} multiplied by a single concentration or two concentrations. Similarly, each reverse rate equation has a form of a reverse rate constant kᵣₑᵥ multiplied by a single concentration or two concentrations. In some embodiments, the concentration or concentrations may be raised to a power, including 2, 3, or a non-integer. The rate equations may be determined based on the stoichiometry of the microstep. In some cases, the rate equations may be based on or adjusted by experimental data or published data. Experimental data may be obtained through experimental system 130. The model of the reaction may include a set of ordinary differential equations, where each ordinary differential equation corresponds to a rate equation.

At block 2315, process 2300 may include simulating an in silico behavior of the reaction. Simulating the in silico behavior of the reaction may be by generating concentrations of the first substrate and the second substrate using the plurality of rate equations (block 2320). For example, the rate constants and the rate equations may be determined from the free energy, and then the rates of change for the substrates and the enzymes may be determined from the rate equations, as explained with FIG. 21, FIG. 16, and FIGS. 12A-12D.

Simulating the in silico behavior of the reaction may also be performed by updating the plurality of initial waypoints to reduce a difference between the in silico behavior of the reaction and an expected behavior of the reaction (block 2325). The free energy of the initial waypoints may be adjusted up or down and then the result of the shift in waypoints may be compared to the result with the initial waypoints. A range of free energies for each waypoint may be used in simulating the in silico behavior. In some embodiments, the difference between the in silico behavior of the reaction and an expected behavior of the reaction may be minimized. The updating of the free energy profile and the comparison with expected behavior may be with a prediction model as described with FIG. 21 and FIG. 16. The reduction of the difference may be by, for example, using a computer system through regression analysis or machine learning techniques. A new free energy profile including the plurality of updated waypoints may be formed. Each of the plurality of updated waypoints may correspond to one of the plurality of initial waypoints. An updated waypoint may correspond to the same end of a component step as an initial waypoint but have a different free energy value.

The difference between the in silico behavior and the expected behavior may be measured based on a comparison between a simulated catalytic rate constant k_{cat} and a simulated Michaelis constant Kₘ of the in silico behavior of the reaction to an expected catalytic rate constant k_{cat} and an expected Michaelis constant Kₘ of the expected behavior of the reaction. The expected catalytic rate constant k_{cat} may be based on an experimentally derived catalytic rate constant k_{cat}. The expected Michaelis constant Kₘ may be based on an experimentally derived catalytic rate constant k_{cat}. The simulated catalytic rate constant k_{cat} and the simulated Michaelis constant Kₘ may be determined from a relationship between the simulated reaction rate and substrate concentrations. The experimentally derived constants may be determined through experimental system 130. The Kₘ and/or k_{cat} (or Vₘₐₓ) may be determined as described with FIG. 14 and FIG. 15. Some embodiments may include methods of determining Kₘ and/or k_{cat} (or Vₘₐₓ), as described in US Application No. XX/XXX,XXX, titled “IN SILICO ENZYMOLOGY,” filed Nov. 9, 2020, the contents of which are incorporated herein by reference for all purposes.

The difference between the in silico behavior of the reaction and an expected behavior of the reaction may be minimized. The difference may be reduced or minimized through machine learning techniques. The minimization may use the prediction model described herein with FIG. 21 and FIG. 16.

A first difference in free energy from a beginning of the initial free energy profile to an end of the initial free energy profile may correspond to an expected change in free energy for the reaction. A second difference in free energy from a beginning of the new free energy profile to an end of the new free energy profile may equal the first difference. The Gibbs free energy change for the reaction as determined in the initial free energy profile may be the same as the Gibbs free energy change for the reaction as determined in the new free energy profile.

In some embodiments, the method may include determining one of the forward rate constant or the reverse rate constant for a rate equation of the plurality of rate equations using the free energy state of an end of a component step and the other rate constant of the forward rate constant or the reverse rate constant. With the new free energy profile, the free energy at each waypoint can be determined. The forward rate constant and the reverse rate constant for the transition between waypoints can then be calculated using the free energy change of this transition. As described with FIG. 21, any combination of forward rate constant and reverse rate constant consistent with a K_{d} (determined from ΔG) at a waypoint may be used. A reverse rate constant or a forward rate consistent may be assumed and the other calculated from K_{d}. In some embodiments, the forward rate constant or the reverse rate constant may be measured or known, and the other rate constant may be calculated from K_{d}.

The simulated behavior may be used as the results of a module or a portion of a module for a partitioned model in block 410 of FIG. 4. Together with other modules, which may also use simulated behavior based on process 2300, a biological system may be simulated.

Some embodiments may also include systems. The system may include one or more data processors. The system may also include a non-transitory computer readable storage medium containing instructions which, when executed on the one or more data processors, cause the one or more data processors to perform part or all of one or more of the methods described herein.

Some embodiments may also include a computer-program product tangibly embodied in a non-transitory machine-readable storage medium, including instructions configured to cause one or more data processors to perform part or all of one or more methods described herein.

After simulating the in silico behavior of the system, a biological system may be engineered or altered based on the simulated behavior. A new gene that may increase the production or activity of an enzyme may be introduced to a biological system (e.g., a cell). The expression of an existing gene in a biological system may be increased or decreased through various genetic or molecular techniques. For example, techniques may include genetic knockout, increased copy number, promoter swap, CRISPRi (inhibition). A particular protein may be engineered to target certain enzymatic properties. These modifications may be introduced in E. coli or other cellular organisms. The behavior of the model may identify certain factors to be adjusted so as to narrow the design space for synthetic biology or metabolic engineering. Process 2300 may include outputting characteristics of an engineered organism having enzymatic activity based on the simulated in silico behavior. For example, the characteristics of the engineered organism may be that the organism has or produces a certain enzyme at a certain concentration with a certain activity. Time and costs for new biological developments, including new drugs and treatments for disorders, may then be reduced. In some embodiments, after simulating the in silico behavior of the system, an experiment (e.g., an in vitro experiment) may be run to verify the simulated behavior.

### VIII. Example Results

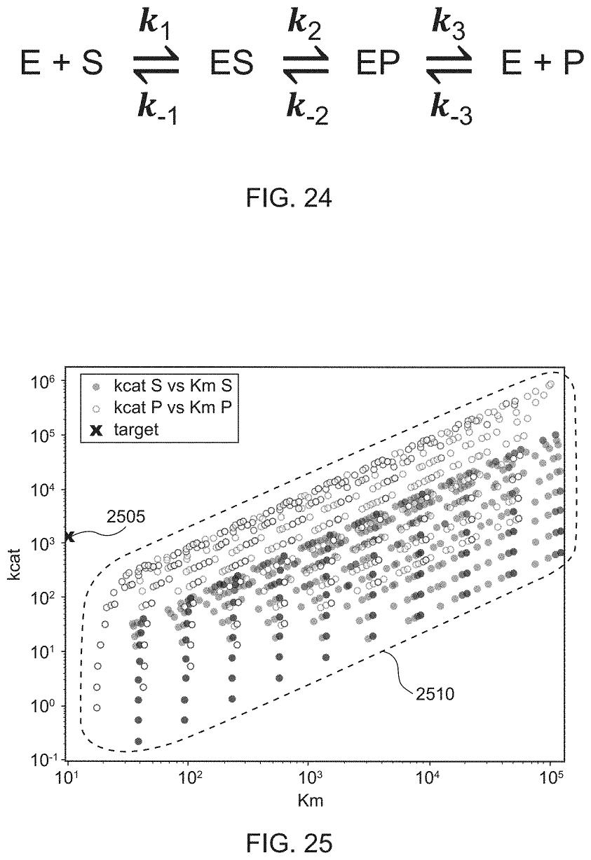

Some embodiments of the present invention result in more accurate results than conventional methods. Conventional methods of solving kinetics for enzymatic reactions may involve using simplifying assumptions. FIG. 24 shows an example enzymatic reaction between a substrate S and an enzyme E to form a product P with the associate rate constants. ES and EP are intermediate complexes.

Conventional assumptions include assuming that ES is more likely to dissociate than to form EP. As a result, k₋₁ is assumed to be much larger than k₂. Additionally, catalysis of ES to EP is assumed to be rate-limiting. Hence, k₃ is assumed to be much larger than k₂. While these assumptions may make sense with some enzymatic reactions, including simpler enzymatic reactions, these assumptions may generate infeasible results in some instances, including for more complicated enzymatic reactions.

FIG. 25 shows a plot of the catalytic rate constant k_{cat} against the Michaelis constant Kₘ for an enzymatic reaction with triose-phosphate isomerase (TPI). The experiment behind FIG. 25 was to systematically generate a large number of free energy profiles and then use in silico enzymology to determine Kₘ and k_{cat} for each profile and plot the results. Target 2505 represents the k_{cat} and the Kₘ from fitting a derived equation of a kinetic model (e.g., similar to FIG. 20) to experimental results (P. Milliard et al., “Metabolic regulation is sufficient for global and robust coordination of glucose uptake, catabolism, energy production and growth in Escherichia coli,” PLoS Computational Biology (2017)). The points in area 2510 denote k_{cat} and Kₘ calculated from different free energy profiles, assuming a microscopic on-rate close to the theoretical maximum of around 10¹⁰ per-molar per-second. Target 2505 is well outside the range of the results in area 2510.

The graph indicates that the k_{cat} and/or Kₘ that are determined from fitting to a derived kinetic model equation is unrealistic given the likely values for the rate constants of microsteps. The graph demonstrates that trying to create a single rate law equation for a system of several microscopic steps may not be possible. A closed-form rate law equation solution for the whole system may not be possible without simplifying assumptions. Simplifying assumptions may result in mathematically and/or thermodynamically infeasible rate constants. The simplifying assumptions may result in k_{cat} and/or Kₘ that do not match with experimentally derived constants. The resulting k_{cat} and/or Kₘ otherwise may be considered feasible without going through a validation using free energy profiles as illustrated in FIG. 25. Some embodiments of the present invention using free energy profiles therefore may provide more accurate results than conventional methods. Some embodiments using free energy profiles do not use the same simplifying assumptions.

### IX. Example Computing Environment

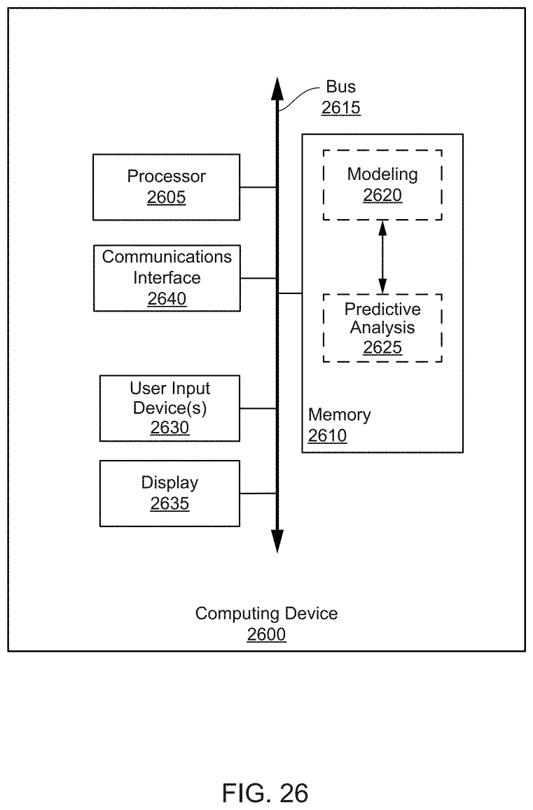

FIG. 26 illustrates an example computing device 2600 suitable for modeling in silico the kinetics of systems of connected biochemical reactions according to this disclosure (e.g., running a module of biological system model 200 described with respect to FIG. 2). The example computing device 2600 includes a processor 2605 which is in communication with the memory 2610 and other components of the computing device 2600 using one or more communications buses 2615. The processor 2605 is configured to execute processor-executable instructions stored in the memory 2610 to perform one or more methods for modeling in silico the kinetics of systems of connected biochemical reactions according to different examples, such as part or all of the example processes 400, 600, and 2300, described herein with respect to FIGS. 1-25. In this example, the memory 2610 stores processor-executable instructions that provide a modeling module 2620 and a predictive analysis module 2625 for one or more reaction pathways, processes, or systems of interest, as discussed herein with respect to FIGS. 1-25.

The modeling module 2620 (e.g., part of the biological system model 200 described with respect to FIG. 2) may be configured to derive a in silico behavior of the system based on the contribution of each component step of the plurality of component steps to the rate of change of the molecules within a pathway, process, or reaction. The predictive analysis module 2625 may be configured to predict a new energy profile, using a prediction model (e.g., prediction model 1610 described with respect to FIG. 16), for the reaction based on the derived in silico behavior of the reaction. Thereafter, the contribution of each component step to the rate of change of the molecules within the pathway, process, or reaction may be applied iteratively over a unit of time back to a state vector. For example, after results have been generated by the modeling module 2620 and the predictive analysis module 2625, a cross-module result synthesizor can access the module-specific results (from one or more module-specific results data stores or direct data availing) and synthesize the results to update high-level data such as a state vector (e.g., stored in a high-level metabolite data store). The state vector may then be used as input to simulate states and reactions of modules configured by an integrated development environment (e.g., the interaction system 100 described with respect to FIG. 1) and/or used by a simulator (e.g., module-specific simulation controller 500 described with respect to FIG. 5) to generate metabolite time-course data according to various embodiments.

The computing device 2600, in this example, also includes one or more user input devices 2630, such as a keyboard, mouse, touchscreen, microphone, etc., to accept user input. The computing device 2600 also includes a display 2635 to provide visual output to a user such as a user interface. The computing device 2600 also includes a communications interface 2640. In some examples, the communications interface 2640 may enable communications using one or more networks, including a local area network (“LAN”); wide area network (“WAN”), such as the Internet; metropolitan area network (“MAN”); point-to-point or peer-to-peer connection; etc. Communication with other devices (e.g., other devices within the interaction system 100 described with respect to FIG. 1) may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

### X. Additional Considerations

Specific details are given in the above description to provide a thorough understanding of some embodiments. However, it is understood that some embodiments can be practiced without these specific details. For example, circuits can be shown in block diagrams in order not to obscure some embodiments in unnecessary detail. In other instances, well-known circuits, processes, algorithms, structures, and techniques can be shown without unnecessary detail in order to avoid obscuring some embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof

Also, it is noted that some embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, some embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A computer-implemented method comprising:
initializing an initial free energy profile for a reaction, the reaction being part of a pathway or process in a system to be modeled, wherein:
the initial free energy profile comprises a plurality of initial waypoints, each initial waypoint representing a free energy state,
the reaction comprises an enzyme, a first substrate, and a second substrate,
the reaction comprises a plurality of component steps,
each end of a component step of the plurality of component steps corresponds to an initial waypoint of the plurality of initial waypoints, and
a first initial waypoint of the plurality of initial waypoints represents a first free energy state of a multi-substrate complex of the enzyme bound to the first substrate and to the second substrate;
generating a plurality of rate equations, each rate equation representing a component step of the plurality of component steps, each rate equation comprising a forward rate constant or a reverse rate constant and one or more concentrations of molecules, the molecules including the enzyme, the first substrate, and the second substrate; and
simulating an in silico behavior of the reaction by:
generating concentrations of the first substrate and the second substrate using the plurality of rate equations, and
updating the plurality of initial waypoints to reduce a difference between the in silico behavior of the reaction and an expected behavior of the reaction, forming a new free energy profile comprising a plurality of updated waypoints.

2. The computer-implemented method of claim 1, wherein:
the difference is measured based on a comparison between a simulated catalytic rate constant k_{cat} and a simulated Michaelis constant Kₘ of the in silico behavior of the reaction to an expected catalytic rate constant k_{cat} and an expected Michaelis constant Kₘ of the expected behavior of the reaction.

3. The computer-implemented method of claim 1, wherein:
a first difference in free energy from a beginning of the initial free energy profile to an end of the initial free energy profile corresponds to an expected change in free energy for the reaction, and
a second difference in free energy from a beginning of the new free energy profile to an end of the new free energy profile equals the first difference.

4. The computer-implemented method of claim 1, wherein:
the plurality of component steps comprises a first binding component step and a second binding component step,
the first binding component step comprises binding between the enzyme and the first substrate to form an enzyme:first substrate complex, and
the second binding component step comprises binding between the enzyme and the second substrate to form an enzyme:second substrate complex.

5. The computer-implemented method of claim 4, wherein:
the plurality of component steps comprises a third binding component step and a fourth binding component step,
the third binding component step comprises binding between the enzyme:first substrate complex and the second substrate to form the multi-substrate complex, and
the fourth binding component step comprises binding between the enzyme:second substrate and the first substrate.

6. The computer-implemented method of claim 5, wherein:
the plurality of component steps comprises a transition component step, a first dissociation component step, a second dissociation component step, a third dissociation step, and a fourth dissociation step,
the transition component step comprises a chemical conversion of the multi-substrate complex to a multi-product complex comprising the enzyme, a first product, and a second product,
the first dissociation component step comprises a dissociation of the multi-product complex into a first complex comprising the enzyme and the first product,
the second dissociation component step comprises a dissociation of the multi-product complex into a second complex comprising the enzyme and the second product,
the third dissociation step comprises a dissociation of the first complex into the enzyme and the first product, and
the fourth dissociation step comprises a dissociation of the second complex into the enzyme and the second product.

7. The computer-implemented method of claim 1, further comprising:
determining one of the forward rate constant or the reverse rate constant for a rate equation of the plurality of rate equations using the free energy state of an end of a component step and the other rate constant of the forward rate constant or the reverse rate constant.

8. The computer-implemented method of claim 1, further comprising:
outputting characteristics of an engineered organism having enzymatic activity based on the simulated in silico behavior.

9. A system comprising:
one or more data processors; and
a non-transitory computer readable storage medium containing instructions which, when executed on the one or more data processors, cause the one or more data processors to perform actions including:
initializing an initial free energy profile for a reaction, the reaction being part of a pathway or process in a system to be modeled, wherein:
the initial free energy profile comprises a plurality of initial waypoints, each initial waypoint representing a free energy state,
the reaction comprises an enzyme, a first substrate, and a second substrate,
the reaction comprises a plurality of component steps,
each end of a component step of the plurality of component steps corresponds to an initial waypoint of the plurality of initial waypoints, and
a first initial waypoint of the plurality of initial waypoints represents a first free energy state of a multi-substrate complex of the enzyme bound to the first substrate and to the second substrate;
generating a plurality of rate equations, each rate equation representing a component step of the plurality of component steps, each rate equation comprising a forward rate constant or a reverse rate constant and one or more concentrations of molecules, the molecules including the enzyme, the first substrate, and the second substrate; and
simulating an in silico behavior of the reaction by:
generating concentrations of the first substrate and the second substrate using the plurality of rate equations, and
updating the plurality of initial waypoints to reduce a difference between the in silico behavior of the reaction and an expected behavior of the reaction, forming a new free energy profile comprising a plurality of updated waypoints.

10. The system of claim 9, wherein:
the difference is measured based on a comparison between a simulated catalytic rate constant k_{cat} and a simulated Michaelis constant Kₘ of the in silico behavior of the reaction to an expected catalytic rate constant k_{cat} and an expected Michaelis constant Kₘ of the expected behavior of the reaction.

11. The system of claim 9, wherein:
a first difference in free energy from a beginning of the initial free energy profile to an end of the initial free energy profile corresponds to an expected change in free energy for the reaction, and
a second difference in free energy from a beginning of the new free energy profile to an end of the new free energy profile equals the first difference.

12. The system of claim 9, wherein:
the plurality of component steps comprises a first binding component step and a second binding component step,
the first binding component step comprises binding between the enzyme and the first substrate to form an enzyme:first substrate complex, and
the second binding component step comprises binding between the enzyme and the second substrate to form an enzyme:second substrate complex.

13. The system of claim 12, wherein:
the plurality of component steps comprises a third binding component step and a fourth binding component step,
the third binding component step comprises binding between the enzyme:first substrate complex and the second substrate to form the multi-substrate complex, and
the fourth binding component step comprises binding between the enzyme:second substrate and the first substrate.

14. The system of claim 13, wherein:
the plurality of component steps comprises a transition component step, a first dissociation component step, a second dissociation component step, a third dissociation step, and a fourth dissociation step,
the transition component step comprises a chemical conversion of the multi-substrate complex to a multi-product complex comprising the enzyme, a first product, and a second product,
the first dissociation component step comprises a dissociation of the multi-product complex into a first complex comprising the enzyme and the first product,
the second dissociation component step comprises a dissociation of the multi-product complex into a second complex comprising the enzyme and the second product,
the third dissociation step comprises a dissociation of the first complex into the enzyme and the first product, and
the fourth dissociation step comprises a dissociation of the second complex into the enzyme and the second product.

15. The system of claim 9, the actions further comprising:
determining one of the forward rate constant or the reverse rate constant for a rate equation of the plurality of rate equations using the free energy state of an end of a component step and the other rate constant of the forward rate constant or the reverse rate constant.

16. A computer-program product tangibly embodied in a non-transitory machine-readable storage medium, including instructions configured to cause one or more data processors to perform actions including:
initializing an initial free energy profile for a reaction, the reaction being part of a pathway or process in a system to be modeled, wherein:
the initial free energy profile comprises a plurality of initial waypoints, each initial waypoint representing a free energy state,
the reaction comprises an enzyme, a first substrate, and a second substrate,
the reaction comprises a plurality of component steps,
each end of a component step of the plurality of component steps corresponds to an initial waypoint of the plurality of initial waypoints, and
a first initial waypoint of the plurality of initial waypoints represents a first free energy state of a multi-substrate complex of the enzyme bound to the first substrate and to the second substrate;
generating a plurality of rate equations, each rate equation representing a component step of the plurality of component steps, each rate equation comprising a forward rate constant or a reverse rate constant and one or more concentrations of molecules, the molecules including the enzyme, the first substrate, and the second substrate; and
simulating an in silico behavior of the reaction by:
generating concentrations of the first substrate and the second substrate using the plurality of rate equations, and
updating the plurality of initial waypoints to reduce a difference between the in silico behavior of the reaction and an expected behavior of the reaction, forming a new free energy profile comprising a plurality of updated waypoints.

17. The computer-program product of claim 16, wherein:
the difference is measured based on a comparison between a simulated catalytic rate constant k_{cat} and a simulated Michaelis constant Kₘ of the in silico behavior of the reaction to an expected catalytic rate constant k_{cat} and an expected Michaelis constant Kₘ of the expected behavior of the reaction.

18. The computer-program product of claim 16, wherein:
a first difference in free energy from a beginning of the initial free energy profile to an end of the initial free energy profile corresponds to an expected change in free energy for the reaction, and
a second difference in free energy from a beginning of the new free energy profile to an end of the new free energy profile equals the first difference.

19. The computer-program product of claim 16, wherein:
the plurality of component steps comprises a first binding component step and a second binding component step,
the first binding component step comprises binding between the enzyme and the first substrate to form an enzyme:first substrate complex, and
the second binding component step comprises binding between the enzyme and the second substrate to form an enzyme:second substrate complex.

20. The computer-program product of claim 19, wherein:
the plurality of component steps comprises a third binding component step and a fourth binding component step,
the third binding component step comprises binding between the enzyme:first substrate complex and the second substrate to form the multi-substrate complex, and
the fourth binding component step comprises binding between the enzyme:second substrate and the first substrate.
